(19) United States
(12) Patent Application Publication
(10) Pub. No.: US 2021/0207135 A1
(43) Pub. Date: Jul. 8, 2021

(54) INHIBITION OF FOLLISTATIN

(71) Applicant: Housey Pharmaceutical Research Laboratories, L.L.C., Southfield, MI (US)

(72) Inventors: Gerard M. Housey, Southfield, MI (US); Monica E. Balash, Farmington Hills, MI (US)

(21) Appl. No.: 17/055,800

(22) PCT Filed: May 17, 2019
(86) PCT No.: PCT/US2019/032969

§ 371 Date: Nov. 16, 2020

Related U.S. Application Data
(60) Provisional application No. 62/673,082, filed on May 17, 2018.

(51) Int. Cl. C12N 15/113; G01N 33/50; G01N 33/563; A61K 45/06

(52) U.S. Cl. CPC C12N 15/113; G01N 33/5044; G01N 2800/52; A61K 45/06; C12N 2310/14; G01N 33/563

(57) ABSTRACT

Provided herein are methods for modulating follistatin, such as inhibiting follistatin, suppressing the production of follistatin, reducing the level of follistatin, inhibiting the function of follistatin, or a combination thereof. The method can include administration of a compound that acts to modulate follistatin. In one embodiment, the compound is administered to a patient having or at risk or having a disease or condition selected from diabetes, pre-diabetes, metabolic syndrome, insulin resistance, dementia, and obesity, and optionally the disease or condition is prevented, treated, ameliorated, or a combination thereof.

### CROSS REFERENCE TO RELATED APPLICATIONS

This application claims the benefit of U.S. Provisional Application No. 62/673,082, filed 17 May 2018, the disclosure of which is incorporated by reference herein in its entirety.

### FIELD

This disclosure comprises a general method for the prevention, induction of long term remission, or cure of various metabolic diseases and disorders in human beings and animals—including obesity, type 2 diabetes, metabolic syndrome, glucose intolerance, insulin resistance and other disorders—by reducing the level of follistatin produced in the body and circulating in the blood.

### BACKGROUND

### Introduction

Diabetes, pre-diabetes, metabolic syndrome and obesity are epidemics in major countries throughout the world. Diabetes is manifest by the loss of the ability to control the amount of sugar (glucose) present in the blood and other life-threatening complications—including dyslipidemia, nonalcoholic fatty liver disease (NAFLD), cardiovascular disease, kidney disease, neuropathy and retinopathy. It has been estimated that one of every five people born after the year 2000 will develop diabetes in their lifetime. More than 16 million Americans already suffer from this disease. In September of 2015, the U.S. Center for Disease Control (CDC) published its findings revealing that from 1988 until 2012, diabetes and prediabetes increased steadily in the U.S., as a direct result of a diet full of refined Sugar-Sweetened Beverages (“SSBs”) and high fat foods, especially fast foods. According to the CDC, 12-14% of the US population is now diabetic, and 34-38% of the population is pre-diabetic. Similar incidence and prevalence rates are found in other “westernized” countries, including Canada, Mexico, Western Europe, and even China. Total costs of diagnosed diabetes in the United States in 2017 was $327 billion (http://www.diabetes.org/diabetes-basics/statistics/).

Normal control of blood glucose is essential for good health and well-being. Blood glucose levels in the human body are maintained within carefully controlled limits due to the effects of insulin on various tissues and organs. When a person eats a meal, blood glucose (sugar) rises as the food and beverages are digested and absorbed. The pancreas responds by producing insulin to control the rise in blood sugar by stimulating insulin-responsive tissues such as fat, liver and muscle to remove excess glucose from the bloodstream, and inhibit production of glucose by the liver. Insulin also has important effects on the function of the cardiovascular system and in the central nervous system. Through this hormone-mediated mechanism, an individual can maintain blood glucose levels within the normal range and avoid progressive metabolic disease and life-threatening cardiovascular events. If the concentration of blood glucose strays outside of the normal limits, as it does in pre-diabetics, metabolic syndrome and untreated diabetic patients, then serious and sometimes fatal consequences can occur.

Diabetes is a complex and life-threatening disease that has been known for more than 2000 years. It occurs in mammals as diverse as monkeys, cats, dogs, rats, mice and human beings. The discovery of insulin and its purification in 1921 for use in people provided a partial treatment for diabetes that is still in widespread use today. Insulin levels are ordinarily adjusted by the body on a moment to moment basis to keep the blood sugar level within a narrow physiological range. Periodic insulin injections, however, can only approximate the normal state because the cellular response to insulin in many cases is also reduced. Consequently, for these and other reasons which will be discussed in detail below, life threatening complications still occur during the lifetime of treated diabetic patients, especially in the case of type 2 (adult-onset) diabetes.

Diabetes arises from various causes, including dysregulated glucose sensing or insulin secretion (Maturity onset diabetes of youth; MODY), autoimmune-mediated. beta-cell destruction (type 1 diabetes), or insufficient compensation for peripheral insulin resistance (type 2 diabetes). (Zimmet, P. et al., 2001). In 2015, approximately 1.25 million American children and adults have type 1 diabetes. However, type 2 diabetes (or “T2D”) is the most prevalent form of the disease, which is closely associated with obesity, usually occurs at middle age, and as shown by the CDC studies discussed above now afflicts more than 30 million Americans. It is increasingly being recognized that obesity, pre-diabetes, metabolic syndrome and ultimately diabetes together comprise a spectrum of progressively worsening morbidity states that eventually lead to a constellation of sequelae, increasing the probability that numerous additional diseases may arise in the afflicted individual. For example, an individual afflicted with obesity, diabetes, pre-diabetes or metabolic syndrome is at a substantially increased risk for the development of atherosclerosis, multiple forms of cancer, dementia, heart disease, non-alcoholic steatohepatitis (NASH) and stroke, as well as other less common diseases and disorders. Key molecular and physiologic markers for identifying individuals at risk for these disorders include higher circulating insulin levels, elevated glucose levels, dyslipidemia, and hypertension.

At the molecular level, diabetes arises from various causes: autoimmune-mediated β-cell destruction (Type 1 Diabetes, or “T1D”); impaired glucose sensing or insulin secretion, peripheral insulin resistance and insufficient β-cell insulin secretory capacity to compensate (Type 2 Diabetes, “T2D”) and Maturity Onset Diabetes of Youth (MODY) (Chen, L. et al., 2012; Lipman, T. H. et al., 2013; Tuomilehto, J. et al., 2013; Yisahak, S. F. et al., 2014; Kendall, D. L. et al., 2014; George, M. M. et al., 2013; Samaan, M. C. et al., 2013; Savoye, M. et al., 2014; Monzavi, R. et al., 2006). T2D is the most prevalent form that typically manifests in middle age (Menke, A. et al., 2015; http://www.diabetes.org/diabetes-basics/statistics). However, T2D is becoming more common in children and adolescents in the developed world (Menke, A. et al., 2015; http://www.diabetes.org/diabetes-basics/statistics).

Physiologic stress, the response to trauma, inflammation, or excess nutrients promote T2D by activating pathways that impair the post-receptor response to insulin in various tissues including the liver, adipose, muscle, vasculature, and others (Hotamisligil, G. S. et al., 2006; Petersen, K. F., et al., 2007; Semple, R. K. et al., 2009). In a few informative cases, mutations in the insulin receptor or AKT2 explain severe forms of insulin resistance (Semple, R. K. et al., 2009). More common forms of T2D are associated with multiple gene variants with modest effects upon glucose homeostasis-including IRS1 (Rung, J. et al., 2009; Kilpeläinen, T. O. et al., 2011), PPARγ, PPAR γ C1A, Kir6.2 (KCNJ11), CAPN10, TCF7L2, adiponectin (ADIPOQ), ADIPOR2, HNF4α, UCP2, SREBF1, or high plasma IL-6 concentrations (Nandi, A. et al., 2004; Vaxillaire, M. et al., 2008). Dysregulated insulin signaling exacerbated by chronic hyperglycemia and compensatory hyperinsulinemia promotes a cohort of acute and chronic sequela (DeFronzo, R. A. et al., 2004; Reaven, G. M. et al., 1995). Untreated diabetes progresses to ketoacidosis (most frequent in T1D) or hyperglycemic osmotic stress (most frequent in T2D), which are immediate causes of morbidity and mortality (Kitabchi, A. E. et al., 2006). Diabetes is also associated with numerous chronic life threatening complications including increased cerebrovascular disease. Similarly, cardiovascular diseases such as peripheral vascular disease, coronary artery disease, hypertension, congestive heart failure, and myocardial infarction are uniformly increased in diabetics as a result of the synergistic effects of hyperglycemia, dyslipidemia, hyperinsulinemia, and other cardiovascular risk factors (Brownlee, M. et al., 2005; Stentz, F. B. et al., 2004). Liver complications including Non Alcoholic Fatty Liver Disease (NAFLD), Non Alcoholic Steatohepatitis (NASH) and increased incidence of liver carcinomas are also observed in diabetics (Herzig, S. et al., 2012; Schattenberg, J. M. et al., 2011; D'Adamo, E. et al., 2013). Diabetes is also associated with degeneration in the central nervous system (Cole, G. M. et al., 2007; Barbieri, M. et al., 2003). Prediabetes is a growing health concern where prevention of disease progression to full-blown diabetes is beneficial (Savoye, M. et al., 2014; Monzavi, R. et al., 2006). As insulin resistance and elevated blood glucose can be detected earlier, offering a safe treatment that can reverse and normalize prediabetic patients offers a potential diabetes cure (Savoye, M. et al., 2014; Monzavi, R. et al., 2006). Treatment of prediabetic adolescents and young adults to stop their progression to diabetes would significantly enhance the quality of their lives and have a significant impact on the lifetime cost of their healthcare. Enhanced IRS2 signaling has the potential to improve glucose metabolism in the liver, enhance peripheral insulin sensitivity, increase insulin secretion, revitalize β-cells, and promote central nervous system control of peripheral metabolism (White, M. F. et al., 2006; Norquay, L. D. et al., 2009; Terauchi, Y. et al., 2007; Housey and White, 2003; Housey and Balash, 2014).

### The Proximal Effects of Insulin Signaling: Insulin Receptor Substrates

Work with transgenic mice suggests that the proximal effects of insulin signaling that give rise to many insulin responses—especially those associated with somatic growth and nutrient homeostasis—are mediated through IRS1 or IRS2 (White, M. F. et al., 2003). The IRS-proteins are adapter molecules that link the insulin-like receptors to common downstream signaling cascades (FIG. 1). Four IRS genes have been identified in rodents, three of which are conserved in humans (IRS1, IRS2 and IRS-4) (Bjornholm, M. et al., 2002). IRS1 and IRS2 proteins are broadly expressed in mammalian tissues, whereas IRS-4 is largely restricted to the hypothalamus and at low levels in a few other tissues (Numan, S. et al., 1999). Each of these IRS proteins is targeted to the activated insulin-like receptors through an NH₂-terminal pleckstrin homology (PH) domain and a phosphotyrosine binding (PTB) domain.

The IRS-proteins bind through their PTB domain to the juxtamembrane autophosphorylation site in the insulin receptor at pY₉₇₂. The pY₉₇₂ resides in a canonical PTB-domain binding motif (NPEpY₉₇₂) (White, M. F. et al., 1988; Eck, M. J. et al., 1996). The juxtamembrane region is about 35 residues long and connects the transmembrane helix of the IRβ subunit to the kinase domain (. Unlike other receptor tyrosine kinases, the insulin receptor kinase is not regulated by autophosphorylation in the juxtamembrane region—although the NPEY-motif can modulate receptor trafficking (Backer, J. M. et al., 1990; Hubbard, S. R. et al., 2004). However, phosphorylation of Tyr₉₇₂ creates a docking site for the phosphotyrosine binding (PTB) domain in the IRS-proteins and SHC (White, M. F. et al., 1988; Pelicci, G. L. et al., 1992). The NPEpY₉₇₂-motif fills an L-shaped cleft on the PTB-domain, while the N-terminal residues of the bound peptide form an additional strand in the β sandwich (Eck, M. J. et al., 1996). The NPEpY₉₇₂-motif is a low-affinity binding site for the PTB domain of IRS1 (Kd ˜87 μM), owing to a destabilizing effect of E₉₇₁ that facilitates autophosphorylation of Y₉₇₂ by the insulin receptor (Farooq, A. et al., 1999; Hubbard, S. R. et al., 2013). By comparison, the PTB domain of SHC binds to NPEpY₉₇₂ with a much higher affinity (K_{d} ˜4 μM).

The pleckstrin homology (PH) domain immediately upstream of the PTB domain helps recruit the IRS-proteins to the insulin receptor ((Yenush, L. et al., 1996). The PH domain is structurally similar but functionally distinct from the PTB domain (Dhe-Paganon, S. et al., 1999). Although the PH-domain promotes the interaction between IRS and the insulin receptor, its mechanism of action remains poorly understood as it does not bind phosphotyrosine. PH domains are generally thought to bind phospholipids, but the PH domains in IRSs are poor examples of this binding specificity (Lemmon, M. A. et al., 1996; Lemmon, M. A. et al., 2002). By contrast, the IRS1/IRS2 PH domain binds to negatively charged sequence motifs in various proteins, which might be important for insulin receptor recruitment (Burks, D. J. et al., 1997). Regardless, the PH domain in the IRS-protein plays an important and specific role as it can be interchanged among the IRS-proteins without noticeable loss of bioactivity. By contrast, substitution of the IRS1 PH domain with heterologous PH-domains from unrelated proteins reduces IRS1 function, which confirms a specific functional role for the IRS1 PH domain (Burks, D. J. et al., 1998).

IRS2 utilizes an additional mechanism to interact with the insulin receptor, which is absent in IRS1. Amino acid residues 591 and 786—especially Tyr₆₂₄ and Tyr₆₂₈—in IRS2 mediate a strong interaction with the activated IR catalytic site (Sawka-Verhelle, D. et al., 1996; Sawka-Verhelle, D. et al., 1997). This binding region in IRS2 was originally called the kinase regulatory-loop binding (KRLB) domain because tris-phosphorylation of the A-loop was required to observe the interaction (Sawka-Verhelle, D. et al., 1996). Structure analysis reveals an essential functional part of the KRLB-domain—residues 620-634 in murine IRS2—that fits into the ‘open’ catalytic site of the insulin receptor (Wu, J. et al., 2008). With the A-loop out of the catalytic site—by autophosphorylation or other means—Tyr₆₂₁ of IRS2 inserts into the receptor ATP binding pocket while Tyr₆₂₈ aligns for phosphorylation. This interaction might attenuate signaling by blocking ATP access to the catalytic site, or it might promote signaling by opening the catalytic site before tris-autophosphorylation. Interestingly, the KRLB-motif does not bind to the IGF1R possibly explaining signaling differences between IR and IGF1R, as well as the receptor hybrids (Wu, J. et al., 2008).

### The Distal Effects of Insulin Signaling: The Downstream Cascades

Insulin activates its receptor tyrosine kinase that in turn phosphorylates the insulin receptor substrates IRS1 and IRS2, which initiate and regulate the insulin signal. Downstream insulin signaling is composed of a highly integrated network, which coordinates multiple tissue-specific signals that control cellular growth, survival and metabolism, and modulate the strength and duration of the signal through diverse feedback cascades (Taniguchi, C. M. et al., 2006). The cascade begins when insulin stimulates tyrosyl phosphorylation of YXXM-motifs in IRS1 and/or IRS2, which directly recruit and activate the class 1A phosphotidylinositide 3-kinase (PI3K) (See FIG. 1). PI3Ks are lipid kinases central to numerous signaling pathways, which are organized into three classes—class I, class II, and class III. The growth factor-regulated class IA PI3Ks are composed of two subunits. The catalytic subunit—p110α (PIK3CA), p110β (PIK3CB) or p110γ (PIK3CD)—is inhibited and stabilized upon association with one of several homologous 85 kDa regulatory subunits encoded by PIK3R1 (p85α) or PIK3R2 (p85β).

The PI(3,4,5)P3 produced by the activated PI3K plays a pivotal role to recruit to the plasma membrane and activate various proteins. A key cascade involves the recruitment of several Ser/Thr-kinases by PI(3,4,5)P3 in the plasma membrane, including PDK1 (3′-phosphoinosotide-dependent protein kinase-1) and AKT (v-akt murine thymoma viral oncogene). The role of IRS-proteins in the PI3K→AKT signaling cascade has been validated in a wide array of cell-based and mouse-based experiments including rodent hepatocytes, muscle and adipose tissue (Taniguchi, C. M. et al., 2005; Dong, X. et al., 2006; Dong, X. C. et al., 2008; Kubota, N. et al., 2008).

AKT is activated by phosphorylation of Thr₃₀₈ in its activation loop by the juxtaposed membrane bound PDK1. AKT isoforms have a central role in cell biology as they regulate by phosphorylation many proteins that control cell survival, growth, proliferation, angiogenesis, blood pressure, glucose influx, liver and muscle metabolism, and cell migration (FIG. 1) (Manning, B. D. et al., 2007; Vanhaesebroeck, B. et al., 2012; Humphrey, S. J. et al., 2013). More than 100 AKT substrates are known and several are especially relevant to insulin signaling—including GSK3α/β (blocks inhibition of glycogen synthesis); AS160 (promotes GLUT4 translocation); the BAD•BCL2 heterodimer (inhibits apoptosis); the FOXO transcription factors (regulates gene expression in liver, β-cells, hypothalamus and other tissues); p21^{CIP1} and p27^{KIP1} (blocks cell cycle inhibition); eNOS (stimulates NO synthesis and vasodilatation); PDE3b (hydrolyzes cAMP); and TSC2 (tuberous sclerosis 2 tumor suppressor) that inhibits mTORC1 (mechanistic target of rapamycin complex 1) (FIG. 1). An unbiased MS/MS approach implicates many more AKT substrates in insulin action suggesting that the majority of PI3K-mediated growth factor (insulin) signaling is coordinated through AKT-dependent mechanisms (FIG. 1) (Humphrey, S. J. et al., 2013).

Forkhead box O (FOXO) subfamily of transcription factors (FOXO1, FOXO3a, FOXO4, and FOXO6) regulate expression of target genes involved in DNA damage repair response, apoptosis, metabolism, cellular proliferation, stress tolerance, and longevity (Calnan, D. R. et al., 2008; van der Horst, A. et al., 2007). FOXOs contain several AKT phosphorylation sites, a highly conserved forkhead DNA binding domain (DBD), a nuclear localization signal (NLS) located just downstream of the DBD, a nuclear export sequence (NES), and a C-terminal transactivation domain (Obsil, T. et al., 2008). AKT mediated phosphorylation of FOXO1, FOXO3a and FOXO4 causes their nuclear exclusion leading to ubiquitinylation and degradation in the cytoplasm. Thus, insulin stimulated tyrosine phosphorylation of IRS1 and/or IRS2 directly controls gene expression through the activation of the PI3K→AKT cascade.

Finally, the IRS1/2→PI3K→AKT1/2 cascade phosphorylates many other proteins that activates the serine kinase complex called mTORC1 (Yecies, J. L. et al., 2011; Wan, M. et al., 2011; White, M. F. et al., 2010; Hagiwara, A. et al., 2012; Tsunekawa, S. et al., 2011). The mTORC1 promotes hepatic lipogenesis by stimulating sterol regulatory element-binding factor-1 (SREBPF1) cleavage and activation, which enhances the expression of lipogenic genes; however, SREBPF1 can inhibit IRS2 expression/function (FIG. 1) (Yecies, J. L. et al., 2011; Wan, M. et al., 2011; Hagiwara, A. et al., 2012; Tsunekawa, S. et al., 2011; Laplante, M. et al., 2009; Astrinidis, A. et al., 2005; Hu, C. et al., 1994; Menon, S. et al., 2012).

### Heterologous Regulation/Dysregulation of the Insulin Signaling Cascade

Insulin resistance—reduced responsiveness of tissues to normal insulin concentrations—is a principle feature of type 2 diabetes that leads to compensatory hyperinsulinemia (Reaven, G. et al., 2004). It also underlies risk factors—including hyperglycemia, dyslipidemia and hypertension—for the clustering of type 2 diabetes with cardiovascular disease, non-alcoholic fatty liver disease, and related maladies (metabolic syndrome) (Biddinger, S. B. et al., 2006). Although numerous genetic and physiological factors interact to produce and aggravate insulin resistance, rodent and human studies implicate dysregulated signalling by the insulin receptor substrate proteins IRS1 and IRS2 as a common underlying mechanism (DeFronzo, R. A. et al., 2009; Karlsson, H. K. et al., 2007). Several mechanisms have been proposed to play a role—including transcriptional regulation, translational control, posttranslational modification and IRS degradation—which can conspire to dysregulate the proximal steps of the insulin signaling cascade and contribute to metabolic disease.

Over a decade of genetic experiments in mice establishes that changes in the relative function of a broad array of insulin signaling components, nutrient sensors, and their downstream metabolic effectors can have profound effects upon insulin sensitivity and nutrient homeostasis (Biddinger, S. B. et al., 2006). While this work is remarkably informative, the complexity of heterologous regulation complicates the identification and design of new strategies for the treatment of insulin resistance and its pathological sequelae. Although the list of insulin signaling components and their interactions continues to grow by functional and genetic approaches, the IRSs retain a special position as the integrating node that coordinates insulin responses in all tissues and cells. Indeed, a 50% reduction in the concentration of the IR, IRS1 and IRS2 achieved by genetic methods causes growth deficits and diabetes in mice (Kido, Y. et al., 2000). Thus, reduced IR→IRS signaling throughout life causes metabolic disease. We are now aware of many heterologous pathways that regulate the concentration and function of these proximal insulin signaling components, but how the dysregulation of these mechanisms contribute to the progression of insulin resistance, metabolic disease and type 2 diabetes in people is not understood.

Over the past 15 years, mouse-based experiments have revealed how mutations in genes that mediate the insulin signal contribute to insulin resistance and diabetes (White, M. F. et al., 2003). Recent studies reveal a variety of factors secreted from adipose tissue that inhibit insulin signaling (FFAs, tumor necrosis factor-alpha (TNFα), and resistin) or factors that promote insulin signaling (adipocyte complement-related protein of 30 kDa (adiponectin) and leptin) (Shimomura, I. et al., 2000; Zick, Y. et al., 2005; Ozcan, U. et al., 2004). Dysregulation of IRS-protein function links inflammatory cytokines to insulin resistance and provides a plausible framework to understand the loss of compensatory β-cell function when peripheral insulin resistance emerges (Shimomura, I. et al., 2000; Zick, Y. et al., 2005; Ozcan, U. et al., 2004; Wellen, K. E. et al., 2005; Aguirre, V. et al., 2000; Giraud, J. et al., 2007). Heterologous signaling cascades can inhibit the insulin signal, at least in part, through Ser/Thr-phosphorylation of IRS-1 and/or IRS-2 (FIG. 1). (Copps, K. D. et al., 2012)

Mice lacking the gene for IRS1 or IRS2 are insulin resistant, with impaired liver metabolic function and peripheral glucose utilization (Kubota, N. et al., 2000; Guo, S. et al., 2009; Withers, D. J. et al., 1998; Previs, S. F. et al., 2000). Both types of knockout mice display metabolic dysregulation, but only the IRS2^{-/-} mice develop diabetes between 8-15 weeks of age owing to a near complete loss of pancreatic β-cells (Withers, D. J. et al., 1998). In models of obese mice, IRS2 expression in the liver is decreased as well (Kubota, N. et al., 2000). This disruption of hepatic IRS2 leads to insulin resistance suggesting that hepatic IRS2 as well as IRS1 are critical for the pathogenesis of systemic insulin resistance (Withers, D. J. et al., 1998).

### Liver-Specific Double Gene Knockouts of IRS1 and IRS2 Upregulate FOXO1 and Increase Follistatin Production by the Liver

The molecular mechanism of peripheral insulin resistance and its modulation by liver function has been investigated further by White and colleagues through the creation of mice harboring liver-specific knockouts of both IRS1 and IRS2 (Dong, X. C. et al., 2008; Cheng, Z. et al., 2009; Tao, R. et al., 2018). An intraperitoneal injection of insulin into ordinary wild-type mice rapidly stimulates Akt phosphorylation and the phosphorylation of Akt substrates, including FOXO1 and GSK3β (Dong, X. C. et al., 2008). However, if both IRS1 and IRS2 are knocked out in the liver, the resulting liver double-knockout mice (LDKO) exhibit striking hepatic insulin resistance, which includes constitutive FOXO1 activation. Both IRS1 and IRS2 must be deleted to uncouple the insulin receptor from the hepatic PI3K→AKT cascade as both IRS-proteins mediate insulin signals in liver (FIG. 1) (Kubota, N. et al., 2008). These results confirm the shared and absolute requirement for IRS1 or IRS2 for hepatic insulin signaling, and demonstrate that loss of both IRS1 and IRS2 in the liver gives rise to constitutive FoxO1 activity (FIGS. 2, 3).

Remarkably, deletion of hepatic IRS1 and IRS2 also causes insulin resistance in peripheral tissues such as white adipose tissue (WAT) by a heretofore unrecognized molecular mechanism. See FIGS. 3A & 3B. (Tao, R. et al., 2018). To understand how hepatic insulin resistance leads to peripheral insulin resistance, the function of dysregulated hepatokine secretion has been investigated, and recent evidence has implicated the binding protein follistatin (Fst) as a key mediator in peripheral insulin resistance, especially in WAT (Tao, R. et al., 2018). Follistatin increases more than 10-fold in LDKO-liver as determined by qPCR, but its levels normalize in LTKO-liver (in which FoxO1 has also been knocked out) and plasma (Tao, R. et al., 2018). The 5′ promoter region of Fst contains FoxO1 binding sites, suggesting that Fst expression can be induced by nuclear FoxO1. Many cells and tissues produce Fst, but most circulating Fst comes from the liver (Hansen, J. S. et al., 2016) See FIGS. 3A & 3B. In mice, two Fst isoforms are generated by alternative mRNA splicing, including membrane-bound (autocrine) Fst288 that contains a functional heparin binding site, and the longer circulating (endocrine) Fst315 that exhibits reduced heparin binding (Lerch et al., 2007).

Fst can neutralize TGFβ-superfamily ligands—including activin, myostatin, BMP2, 4, 6, 7, 11 and BMP15. TGFβ-superfamily signaling begins when the ligand binds to and activates its congnate heteromeric receptor serine kinase, composed of two ‘type II’ and two ‘type I’ receptors, which phosphorylate Smads to regulate gene expression (See FIG. 2). Fst can regulate ligand interactions at the receptor positively or negatively, so the exact physiologic role of Fst to date has been uncertain (Hansen, J. S. et al., 2016; Han, H. Q. et al., 2013). Since Fst is induced by exercise, inflammation, or glucagon during starvation, it might link systemic nutrient and energy homeostasis with TGFβ-regulated gene expression, growth and differentiation (Hansen, J. S. et al., 2016). Fst is moderately elevated in plasma of insulin resistant and hyperglycemic T2DM patients (Hansen, J. et al., 2013). Interestingly, overexpression of Fst promotes insulin resistance—yet preserves β-cell function in the diabetic pancreas by promoting β-cell proliferation (Zhao, C. et al., 2015; Ungerleider, N. A. et al., 2013). Chronically upregulated FoxO1→Fst in LDKO-mice promotes metabolic disease by exacerbating peripheral insulin resistance, hyperinsulinemia and liver failure. Thus, regardless of Fst's ultimate mechanism of action, therapeutic efficacy for a wide variety of metabolic disorders as discussed above is to be achieved, as this disclosure teaches, through controlled reduction of follistatin activity or levels, or both. This is a concept which stands in contrast to current thinking in the field) (Zhang, L. et al., 2018; Pervin, S. et al., 2017; Singh, R. et al., 2014).

### BRIEF SUMMARY

Since the discovery of Insulin in 1921 by Banting and Best, the molecular mechanism of peripheral insulin resistance, especially in Type 2 diabetes, has remained poorly understood. The inventors have identified the molecular mechanism in LDKO mice (lacking both liver IRS1 and IRS2) that is responsible for inducing peripheral insulin resistance. The inventors and their colleagues have been working on insulin mediated signal transduction targets for more than 15 years, and are aware that when two key related members of the Insulin Receptor Substrate family (IRS1 and IRS2) undergo organ-specific deletion in the liver of a mouse, the molecular response that is generated gives rise to the constitutive activation of the FOXO1 transcription factor (Dong, X. C. et al., 2008; Cheng, Z. et al., 2009). In addition to the effects of elevated FOXO1 activity in the insulin resistant hepatocytes, these mice also develop peripheral insulin resistance, especially in White Adipose Tissue (WAT). Since FOXO1 activates numerous genes (and inhibits others), recent work has studied the profile of genes that are activated or inhibited when FOXO1 expression is elevated (Dong, X. C. et al., 2008). It is now known that increased hepatic FOXO1 activity in LDKO mice leads to the increased production by the liver of a protein termed follistatin (Fst) (Tao, R. et al., 2018).

The inventors have conceived and recognized the therapeutic potential of these recent findings that implicate follistatin (Fst), a circulating binding protein, in the development of insulin resistance. Current ideas in the field have supported the concept that the selective administration of Fst, thereby increasing the level of Fst in a human being, may provide a therapeutic benefit (Zhang, L. et al., 2018; Pervin, S. et al., 2017; Singh, R. et al., 2014). However, from the perspective of the metabolic disorders mentioned above, including diabetes and obesity, the inventors have recognized that a therapeutically effective reduction in the level and/or biological activity of Fst would be beneficial to human beings and other mammals with certain metabolic disorders. Compositions of the disclosure capable of reducing Fst levels or bioactivity (or both) in a human being or other mammal include antibodies (both polyclonal and monoclonal), antibody fragments such as Fab′, nanobodies, other classes of polypeptides such as binding antagonists (inhibitors), nucleic acids, and compounds such as small molecules that disrupt Fst binding to one or more of its target binding partners. Any of the aforementioned substances will, if created and selected according to the teachings of the disclosure, exhibit anti-Fst therapeutic efficacy through one or more of the following mechanisms of action: inhibition of the biological functioning of Fst protein; reduction of its signaling potential; blockade of pathways that produce the Fst protein, including interference with Fst mRNA function; activation of pathways that promote Fst protein degradation or Fst mRNA degradation.

Individuals who are obese as well as those already exhibiting symptoms of pre-diabetes, metabolic syndrome or Type 2 Diabetes, have relatively higher circulating levels of Fst. However, if such patients undergo gastric bypass surgery leading to a successful outcome that includes weight loss and corresponding resolution of the insulin resistance or diabetes that was present pre-operatively, then such patients also show a corresponding fall in Fst levels (Tao et al., 2018; Perakakis et. al., 2019). Thus, the inventors have recognized that selective reduction of Fst (as opposed to its administration) in a mammal in need thereof, would be therapeutically effective at treating a variety of metabolic disorders, including obesity and diabetes.

Evidence further suggests that the insulin receptor substrate (IRS) protein family is of central importance in mediating the effects of insulin on responsive cells and in keeping Fst levels under control during normal physiologic circumstances in a mammal.

Disclosed herein is a method of treating a Fst mediated disease or condition comprising administering an effective amount of a pharmaceutical composition described herein to a subject in need thereof. In certain embodiments, the Fst mediated disease or condition is diabetes, pre-diabetes, metabolic syndrome, insulin resistance, dementia, or obesity. In certain embodiments, the method further comprises administering an antidiabetic agent, insulin, metformin, exenatide, vildagliptin, sitagliptin, a DPP4 inhibitor, meglitinide, exendin-4, liraglutide, dulaglutide, or a GLP1 agonist. The pharmaceutical composition disclosed herein may be administered in a separate pharmaceutical formulation from the antidiabetic agent, insulin, metformin, exenatide, vildagliptin, sitagliptin, a DPP4 inhibitor, meglitinide, exendin-4, liraglutide, or GLP1 agonist. Alternatively, the pharmaceutical composition disclosed herein may be administered in the same pharmaceutical formulation as the antidiabetic agent, insulin, metformin, exenatide, vildagliptin, sitagliptin, a DPP4 inhibitor, meglitinide, exendin-4, liraglutide, dulaglutide, a sodium-glucose transporter type 2 (SGLT-2) inhibitor such as empagliflozin, canagliflozin, or dapagliflozin, or a GLP1 agonist. In certain embodiments, the pharmaceutical composition is administered orally twice per day, 30-60 minutes before meals.

Disclosed herein is a method of inhibiting Fst in a subject in need thereof comprising administering to the subject an effective amount of the pharmaceutical compositions described herein. The term “inhibiting Fst” includes, but is not limited to, reducing expression of Fst in a patient, reducing the amount of Fst in a patient (e.g., the amount in the blood or a cell of a patient), and/or reducing the activity of Fst in a patient (e.g., the activity in the blood or a cell of a patient).

Disclosed herein is a method of inhibiting Fst comprising contacting a cell with the pharmaceutical compositions described herein.

This disclosure provides compounds and methods of providing nutritional support, preventing, inducing durable long-term remission, or curing a patient with diabetes, a metabolic disorder, a central nervous system disease, obesity, fertility, and other human disorders as discussed herein. The disclosure is particularly concerned with the follistatin and with inhibition of Fst-mediated cellular signaling pathways as a mechanism for treating human disease and/or providing beneficial nutritional support.

The disclosure also provides methods of preventing, treating, or ameliorating a Fst mediated disease or condition comprising identifying a patient in need, and administering a therapeutically effective amount of a compound alone or together with a pharmaceutically acceptable salt, ester, amide, or prodrug thereof. A patient in need of prevention, treatment, or amelioration is a patient having or at risk of having of a disease or condition described herein. Fst mediated diseases or conditions include, without limitation, diabetes (type 1 and type 2), insulin resistance, metabolic syndrome, dementia, Alzheimer's disease, hyperinsulinemia, dyslipidemia, and hypercholesterolemia, obesity, hypertension, retinal degeneration, retinal detachment, Parkinson's disease, cardiovascular diseases including vascular disease, atherosclerosis, coronary heart disease, cerebrovascular disease, heart failure and peripheral vascular disease in a subject.

The disclosure also provides for coadministration of a compound alone or together with a pharmaceutically acceptable salt, ester, amide, prodrug, or solvate, to a subject in combination with a second therapeutic agent or other treatment.

Second therapeutic agents for treatment of diabetes and related conditions include biguanides (including, but not limited to metformin), which reduce hepatic glucose output and increase uptake of glucose by the periphery, insulin secretagogues (including but not limited to sulfonylureas and meglitinides, such as repaglinide) which trigger or enhance insulin release by pancreatic β-cells, and PPARγ, PPARα, and PPARα/γ modulators (e.g., thiazolidinediones such as pioglitazone and rosiglitazone).

Additional second therapeutic agents include GLP1 receptor agonists, including but not limited to GLP1 analogs such as exendin-4, liraglutide, dulaglutide, and agents that inhibit degradation of GLP1 by dipeptidyl peptidase-4 (DPP-4). Vildagliptin and sitagliptin are non-limiting examples of DPP-4 inhibitors.

Still other second therapeutic agents include the sodium glucose transporter type 2 (SGLT-2) inhibitors, which reduce the ability of the kidney to reabsorb glucose after it passes through the glomerulus and into the nephron. SLGT-2 inhibitors including, but not limited to empagliflozin, canagliflozin, or dapagliflozin inhibit reabsorption of glucose by the nephron resulting in large amounts of glucose remaining in the urine. This class of compounds has a significant blood glucose lowering effect but also markedly increases the likelihood of bladder infections and pyelonephritis due to the resulting glucosuria.

In certain embodiments of the disclosure, compounds are coadministered with insulin replacement therapy.

According to the disclosure, compounds are coadministered with statins and/or other lipid lowering drugs such as MTP inhibitors and LDLR upregulators, antihypertensive agents such as angiotensin antagonists, e.g., losartan, irbesartan, olmesartan, candesartan, and telmisartan, calcium channel antagonists, e.g. lacidipine, ACE inhibitors, e.g., enalapril, and β-andrenergic blockers (β-blockers), e.g., atenolol, labetalol, and nebivolol.

In another embodiment, a subject is prescribed a compound of the disclosure in combination with instructions to consume foods with a low glycemic index.

In a combination therapy, the compound is administered before, during, or after another thereapy as well as any combination thereof, i.e., before and during, before and after, during and after, or before, during and after administering the second therapeutic agent. For example, a compound of the disclosure can be administered daily while extended release metformin is administered daily (Diabetes Prevention Program Research Group, 2002; Campbell 2007). In another example, a compound of the disclosure is administered once daily and while exenatide is administered once weekly. Also, therapy with a compound of the disclosure can be commenced before, during, or after commencing therapy with another agent. For example, therapy with a compound of the disclosure can be introduced into a patient already receiving therapy with an insulin secretagogue. In addition, compounds of the present disclosure may be administered once or twice daily in conjuction with other nutritional supplements, vitamins, nutraceuticals, or dietary supplements. Examples include GCE, chlorogenic acid, chicoric acid, cinnamon and various other hydroxycinnamic acids, chromium, chromium picolinate, a multivitamin, and so on.

In another aspect, the present disclosure provides pharmaceutically acceptable compositions which comprise a therapeutically-effective amount of one or more of the compounds of the present disclosure, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

In another aspect, the present disclosure provides nutritionally beneficial or supportive compositions which comprise a nutritionally beneficial or supportive amount of one or more of the compounds of the present disclosure, formulated together with one or more active or inactive ingredients carriers (additives) and/or diluents. As described in detail below, the nutritional supplement formulations of the present disclosure may be specially formulated for administration in solid or liquid form, including those adapted for the following: (1) oral administration, for example, drinks, foods, chewable pastes or gums, drenches (aqueous or non-aqueous solutions or suspensions), capsules, tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “effective amount” as used herein means that amount of a compound, material, or composition comprising a compound of the present disclosure which is effective for producing some desired effect in at least a sub-population of cells (e.g., liver cells) in an animal, such as reducing expression of Fst, reducing the amount of Fst, and/or reducing the activity of Fst. The phrase “therapeutically-effective amount” as used herein means that amount of a compound, material, or composition comprising a compound of the present disclosure which is effective for producing some desired therapeutic effect in at least a sub-population of cells (e.g., liver cells) in an animal at a reasonable benefit/risk ratio applicable to any medical treatment, e.g. reasonable side effects applicable to any medical treatment.

The phrase “pharmaceutical composition” necessarily includes, when appropriate, compounds of the disclosure, and the like.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified compound of the disclosure in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (Berge et. al., 1977).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present disclosure may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et. al., 1977).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound of the present disclosure. In certain embodiments, an aforementioned formulation renders orally bioavailable a compound of the present disclosure.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical and nutraceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the herein-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present disclosure are administered as pharmaceuticals, nutraceuticals, or nutritional supplements to humans and animals, they can be given per se or as a composition containing, for example, 0.1 to 99% (more preferably, 10 to 30%) of active ingredient in combination with a pharmaceutically acceptable carrier.

The preparations of the present disclosure may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical or nutritional composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound which is the lowest dose effective to produce a therapeutic or nutritionally supportive effect. Such an effective dose will generally depend upon the factors described herein. Generally, oral, intravenous, intracerebroventricular and subcutaneous doses of the compounds of this disclosure for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present disclosure to be administered alone, it is preferable to administer the compound as a pharmaceutical formulation, both of which are termed “compositions” herein.

The compounds according to the disclosure may be formulated for administration in any convenient way for use in human or veterinary medicine, by analogy with other pharmaceuticals.

### BRIEF DESCRIPTION OF THE FIGURES

FIG. 1 depicts components of the IRS signaling cascade. Insulin regulated PI3- kinase—→PDK1→>Akt and Grb2/Sos→ras kinase cascades. Insulin (INS) stimulates tyrosine phosphorylation of IRS-proteins (pY) that promotes PI3K [p85•p110] and Grb2/SOS binding. Grb2/SOS stimulates the ras→>MAPK (ERK1/2) cascade, which stimulates transcription factors. The PI3K produces PI3,4P₂ and PI3,4,5P₃ (antagonized by the action of PTEN or SHIP2), which recruits PDK1 and AKT to the plasma membrane where AKT1 is activated by phosphorylation at T308 by PDK1 and S473 by mTORC2. AKT phosphorylates many cellular proteins including TSC2 that inhibits a Rheb-specific GTPase that activates mTORC1-dependent protein and activation of SREBP1c, which stimulates lipogenic gene expression. AKT-mediated phosphorylation of FOXO1 results in cytoplasmic sequestration. Akt, mTORC1 and S6K1, mediate ‘homologous’ feedback inhibition of IRS-dependent signaling by Ser/Thr phosphorylation of IRS1/2, while circulating factors (TNFα) activate ‘heterologous’ pathways (Jnk et al.) that phosphorylate IRS on S/T-sites.

FIG. 2 depicts aspects of disruption of the IRS signaling cascade that lead to follistatin dysregulation in the liver. A mechanism of insulin signaling and heterologous dysregulation by Fst and Fgf21. Insulin (Ins) stimulates IRS→PI3K to produce PI3,4 that recruits AKT to the membrane where it is phosphorylated at T308 by PDK1 and S473 by mTORC2. pAKT phosphorylates and inhibits TSC2, FoxO1,GSK3β—and activates PDE3β, and others. Nuclear FoxO1 during insulin resistance increases Fst (follistatin), which inhibits TGFβ-superfamily ligands; and reduces Fgf21. Due to the negative regulatory effect of AKT on FoxO1, deletion of IRS1 and IRS2 leads to loss of AKT activation by PDK1, resulting in the release of the inhibitory phosphorylation of FoxO1 by AKT. The resulting rise in FoxO1 activity leads to marked increases in the expression of Fst in the liver leading to Increased circulating Fst levels in blood and thus generating peripheral insulin resistance in White Adipose Tissue (WAT). Darker gray circles/arrows inhibit; lighter gray circles/arrows activate.

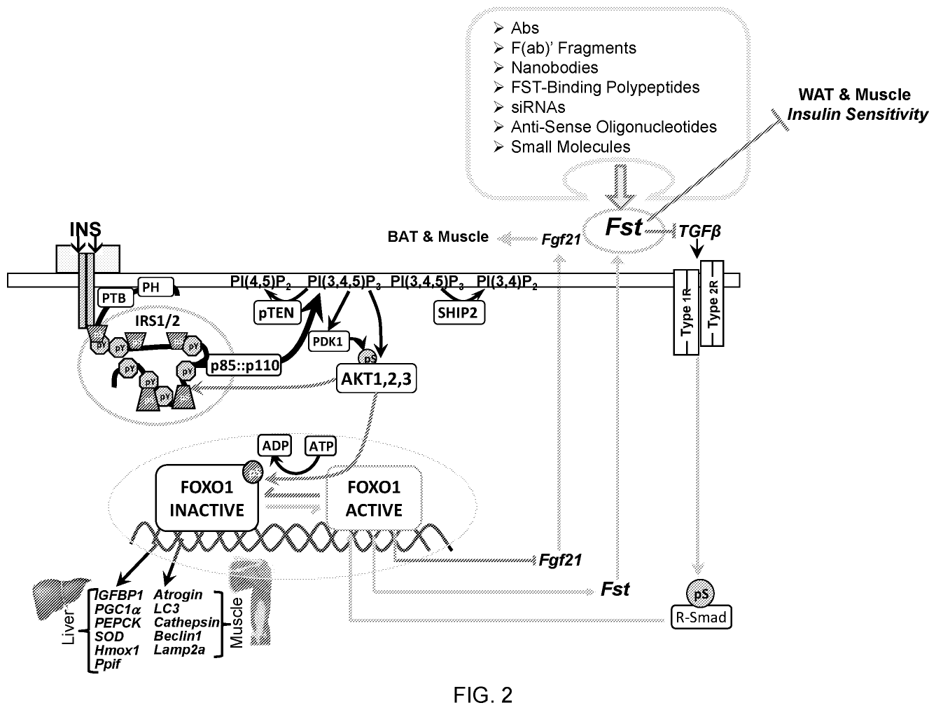

FIG. 3 depicts the relative effects of FoxO1 activity and its relationship to follistatin production and secretion by the liver, which then circulates through the bloodstream and induces insulin resistance in peripheral tissues such as white adipose tissue (WAT). Simplified schematic versions of the normal Fst regulatory physiology in the liver and the deleterios effects that occur with loss of insulin signaling when IRS1 and IRS2 are disrupted in the liver (B). Under normal circumstances, activation of AKT through insulin-mediated activation of IRS1/2 leads to inactivation of FoxO1 by phosphorylation and reduces FST production by the liver. Liver-specific deletion of IRS1 and IRS2 leads to loss of AKT activation by PDK1. This results in loss of the inhibitory phosphorylation of FoxO1 by AKT. The resulting rise in FoxO1 activity leads to marked increases in the expression of Fst in the liver leading to Increased circulating Fst levels in blood and thus generating peripheral insulin resistance in White Adipose Tissue (WAT) as shown in Panel A. Various modalities for therapeutic intervention to reduce Fst-mediated Insulin Resistance in WAT are shown in Panel B. Darker gray circles/arrows inhibit; lighter gray circles/arrows activate.

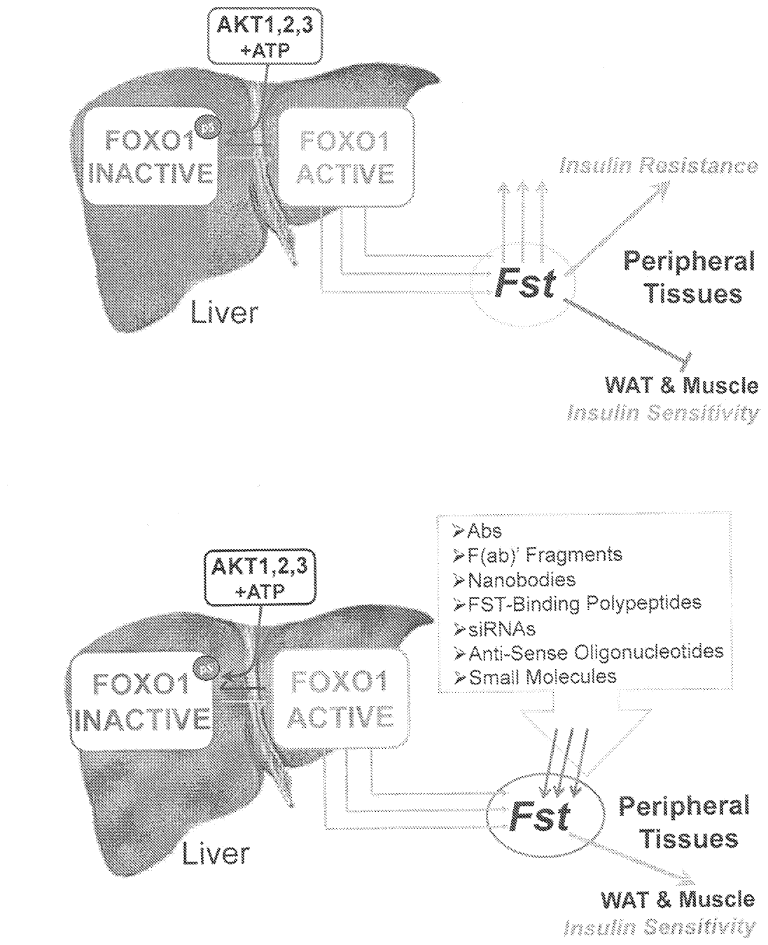

### DETAILED DESCRIPTION

This disclosure pertains to generalized methods of preventing, curing or inducing durable long-term remissions in patients with diabetes, metabolic disorders, central nervous system diseases, obesity, fertility and other human disorders in which an inappropriate level or functional activity of one or more follistatin variants contributes to the disease state. The disclosure is particularly concerned with follistatin and modulation of the activity of follistatin-mediated cellular signaling pathways as a mechanism for treating human disease due to its excessive production in the body and secretion into the circulation under certain conditions.

The disclosure is based on the recognition that the follistatin branch of the insulin/IGF signaling system coordinates important biochemical reactions and signaling pathways needed for proper function of peripheral insulin sensitive tissues and cells (especially in muscle and fat).

Experiments in genetically altered mice that lack follistatin or overexpress Fst reveal the essential role for Fst in peripheral insulin action and the role of Fst in the function, growth and survival of the organism. Dysregulation of Fst signaling, especially excess Fst expression or function at peripheral insulin sensitive tissues, including WAT and liver causes insulin resistance, excess hepatic glucose production and systemic glucose intolerance. Conversely, inhibition of the biological functioning of Fst or reducing its signaling potential, or blocking pathways that produce the protein or activating pathways that promote its degradation correct these problems.

Accordingly, the disclosure is directed to a general method for the treatment, cure, or prevention of various metabolic and related disorders, including diabetes, by reducing the level or functional activity of follistatin in a mammal in need thereof.

In one embodiment, the disclosure is directed to restoring or enhancing insulin sensitivity in a cell by reducing follistatin levels or activity. According to the disclosure, a disease or disorder characterized by elevated levels of follistatin can be treated by reducing follistatin levels or activity (or both). Such diseases include, but are not limited to metabolic disease, diabetes, dyslipidemia, obesity, female infertility, central nervous system disorders, Alzheimer's disease, and disorders of angiogenesis.

In another embodiment of the disclosure, upregulation of IRS2 function (Housey and White; 2003; Housey and Balash; 2014) can reduce Fst and improve WAT and peripheral insulin sensitivity. This would include activation of IRS2 or a complex that includes IRS2. Upregulation of IRS2 function is also accomplished by inhibition of phosphorylation of carboxy terminal serine residues of IRS2. Upregulation of IRS2 function can be accomplished by enhanced expression of IRS2 or by inhibition of degradation of IRS2. Increasing the expression and/or function of IRS2 will lead to a reduction in hepatic Fst levels and a concomitant reduction in the amount of hepatic Fst secreted into the circulation, which will thus improve WAT and peripheral insulin sensitivity and metabolic regulation.

In another embodiment, the disclosure is directed to a method of determining whether a compound is an inhibitor of Fst. In a cell-based assay, a Test Cell is provided which overproduces Fst and exhibits an increase in binding of an Fst-binding protein to Fst—including a specific antibody that binds to Fst—relative to a Control cell which produces Fst at a lower level, or does not produce Fst at all, and which exhibits a lesser amount of binding of said protein to Fst. Small molecules that inhibit Fst are identified by measuring the amount of the Fst binding protein bound to Fst.

In another embodiment, the disclosure is directed to a method of identifying a compound capable of reducing the level of expression from an Fst promoter in a mammalian cell. In one such embodiment, a Test Cell is constructed which contains a construct comprising an Fst promoter operably linked to a reporter gene such that increased expression of the Fst promoter sequence using a substance known to be capable of upregulating the endogenous Fst gene results in an increase in a measurable characteristic of the Test cell resulting from increased expression of the reporter gene (and a corresponding increase in production of the reporter protein. Small molecules that inhibit Fst expression are identified by detecting a decrease in reporter gene activity (reporter protein production).

In another embodiment, the disclosure is directed to a method of identifying a compound capable of interfering with the function of Fst protein to promote WAT insulin resistance. In one such embodiment, a Test Cell—for example a differentiated 3T3L1-adipocyte—is employed to screen for compounds that reverse the effect of serum from insulin-resistant mice containing Fst to promote insulin resistance. An ideal source of Fst-containing serum would be the insulin resistant LDKO-mice, which specifically lack hepatic Irs1 and Irs2. Alternatively, serum from insulin resistant mice overexpressing Fst in the liver can be used. In one embodiment the interaction of IRS1 with the p110 catalytic subunit of PI3K is measured in 3T3-L1 adipocytes exposed to the mouse serum from LDKO-mice. Compounds added to insulin stimulated 3T3-L1 adipocytes incubated with serum from LDKO-mice that increase the association between IRS1 and p110—that is form more IRS1•p110 complex during insulin stimulation—will be identified as compounds that inhibit Fst function.

In another embodiment an increase in insulin-stimulated phosphorylation of AKT is used to identify molecules that inhibit the function of Fst in 3T3-L1 adipocytes exposed to serum from insulin resistant LDKO-mice and lead to better insulin sensitivity through IRS1/IRS2→PI3K→AKT cascade. In another embodiment insulin stimulated dephosphorylation of hormone-sensitive lipase (HSL) is used to identify molecules that inhibit the function of Fst and lead to better insulin sensitivity through IRS1/IRS2→PI3K→AKT→PDE cascade in 3T3-L1 adipocytes incubated with serum from insulin resistant LDKO mice or other mice specifically designed to express and secrete hepatic Fst. Thus compounds that promote IRS1•p110 complex formation, AKT phosphorylation, and/or HSL dephosphorylation in 3T3-L1 adipocytes or other cells incubated with serum from insulin resistant LDKO-mice with elevated circulating Fst reveal inhibitors of Fst function. Compounds identified by these embodiments of this disclosure are insulin sensitizing molecules for the treatment of metabolic disease, diabetes and its related disorders.

For the purposes of this disclosure, the following terms are defined as given below.

“Follistatin”, “follistatin”, “Fst”, an “Fst polypeptide” and an “Fst protein” refer to any isoform of a follistatin protein. Fst proteins are described herein. As used herein, the term “follistatin” or “fst”: refers to the secretory or membrane retained protein that binds activin or other TGFβ superfamily ligands. Follistatin includes Fst, Fst288, Fst303, Fst315, Fst317, Fst344, or any other form generated from alternative splicing of the Fst gene that retains function in a mammal.

“Fst” 16665837 or “Fst gene” or “Fst mRNA” refer to a nucleotide sequence encoding the follistatin (Fst) protein,

As used herein, the terms “inhibitor” and “antagonist” of Fst are used interchangeably, wherein “Fst” and “Fst protein” are identical.

An “inhibitor of follistatin”, which is identical to an “inhibitor of Fst”, is meant to include a compound that binds to Fst alone and reduces the level of Fst or inhibits the function of Fst, or a compound that binds to a complex comprising Fst and other Fst binding partner(s) (Fst “binding partners” include proteins such as myostatin, activin, and other non-proteinaceous molecules that bind to Fst) and wherein said compound cannot bind to the non-Fst binding partner(s) in the absence of Fst.

An “inhibitor of follistatin expression”, which is identical to an “inhibitor of Fst expression” is meant to include a compound that inhibits the expression of the Fst gene by any mechanism, including interference with the production of functional Fst mRNA or enhancing degradation of Fst mRNA.

For this disclosure, to state that a substance “inhibit(s)” follistatin means:the substance can bind to follistatin and reduce follistatin's activity in a cell, a tissue, the blood, or presence in the body; the substance can reduce or eliminate follistatin's functioning; the substance can reduce the amount or level of follistatin; and/or the substance can reduce the expression or production of follistatin. In order for a compound to “inhibit follistatin” or “inhibit Fst”, said compound must be either an inhibitor of Fst or an inhibitor of Fst expression.

Unless explicitly stated otherwise, an “inhibitor”, an “antagonist” and an “inhibitor of follistatin” are also synonymous. The inhibition by an inhibitor may be partial or complete. The terms “bind(s),” “binding,” and “binds to” have their ordinary meanings in the field of biochemistry in terms of describing the interaction between two substances (e.g., enzyme-substrate, protein-DNA, receptor-ligand etc.). As used herein, the term “binds to” is synonymous with “interacts with” in the context of discussing the relationship between a substance and its corresponding target protein or nucleic acid.

As used herein, the terms “compound” and “substance” are used interchangeably, and both terms refer to chemical agents and biological agents.

As used herein, the term “chemical agent” refers to substances that have a molecular weight up to, but not including, 2000 atomic mass units (Daltons). Such substances are sometimes referred to as “small molecules.”

As used herein, “biological agents,” are molecules which include proteins, polypeptides, and nucleic acids, and have molecular weights equal to or greater than 2000 atomic mass units (“amu” or “Daltons”), but not to exceed 990,000 amu.

As used herein, the term “antibody” refers to a protein or immunoglobulin produced in response to an antigen and can “specifically bind” the antigen. An antibody that “specifically binds” an antigen is one that interacts only with the epitope of the antigen that induced the synthesis of the antibody, or interacts with a structurally related epitope. An antibody that “specifically binds” to an epitope will, under the appropriate conditions, interact with the epitope even in the presence of a diversity of potential binding targets.

As used herein, the term “antigen” refers to the protein or peptide target having the epitope to which an antibody specifically binds.

As used herein, the term “fragment” refers to a portion of a polypeptide or polynucleotide. In one embodiment, a fragment retains the activity of the polypeptide or polynucleotide.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Conditions that are “suitable” for an event to occur, or “suitable” conditions are conditions that do not prevent such events from occurring. Thus, these conditions permit, enhance, facilitate, and/or are conducive to the event.

As used herein, “providing” in the context of a composition, an antibody, a nucleic acid, or a small molecule means making the composition, antibody, nucleic acid, or small molecule, purchasing the composition, antibody, nucleic acid, or small molecule, or otherwise obtaining the composition, antibody, nucleic acid, or small molecule.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

According to the present disclosure, a therapeutically effective amount of one or more compounds/substances that inhibit, for instance, the function or level of expression of follistatin protein (Fst) is administered to a mammal in need thereof. The term “mammal” as used herein is intended to include, but is not limited to, humans, laboratory animals, domestic pets and farm animals.

### Fst Proteins

The human FST gene includes six exons spanning 5329 bp on chromosome 5q11.2 and gives rise to two main transcripts of 1122 bp (transcript variant FST344) and 1386 bp (transcript variant FST317) (Grusch, M., 2010). The first exon encodes the signal peptide, the second exon the N-terminal domain and exons 3-5 each code for a follistatin module. Alternative splicing leads to use of exon 6A, which codes for an acidic region in FST344, or exon 6B, which contains two bases of the stop codon of FST317 (Shimasaki, S. et al., 1988).

Mature secreted follistatin protein exists in three main forms consisting of 288, 303, and 315 amino acids (Sugino, K. et al., 1993). The FST344 transcript gives rise to a protein precursor of 344 amino acids, which results in the mature 315 amino acid form (Fst315) after removal of the signal peptide. A fraction of Fst315 is further converted to the 303 amino acid form (Fst303) by proteolytic cleavage at the C-terminus. Signal peptide removal of FST317 leads to the mature 288 amino acid form of follistatin (Fst288). All forms of follistatin contain three follistatin domains (FSD) characterized by a conserved arrangement of 10 cysteine residues. The N-terminal subdomains of the FSD have similarity with EGF-like modules, whereas the C-terminal regions resemble the Kazal domains found in multiple serine protease inhibitors.

In one embodiment, the FST that is modulated is Fst315. An example of a mature human Fst315 protein is as follows:

(SEQ ID NO: 1)
GNCWLRQAKNGRCQVLYKTELSKEECCSTGRLSTSWTEEDVN
DNTLFKWMIFNGGAPNCIPCKETCENVDCGPGKKCRMNKKNK
PRCVCAPDCSNITWKGPVCGLDGKTYRNECALLKARCKEQPE
LEVQYQGRCKKTCRDVFCPGSSTCWDQTNNAYCVTCNRICPE
PASSEQYLCGNDGVTYSSACHLRKATCLLGRSIGLAYEGKCI
KAKSCEDIQCTGGKKCLWDFKVGRGRCSLCDELCPDSKSDEP
VCASDNATYASECAMKEAACSSGVLLEVKHSGSCNSISEDTE
EEEEDEDQDYSFPISSILEW.
(amino acids 30-344 of Genbank accession
number P19883.2)

An example of a follistatin precursor is available at Genbank accession number AAA35851.

An example of polynucleotide sequence encoding a mature human Fst315 protein is available at Genbank accession number AH001463.

### Production of Fst Inhibitors

Inhibitors of the disclosure are prepared using a variety of approaches which are standard in the field and known to the skilled practitioner. Following the creation of such inhibitors, testing of the inhibitor for potential therapeutic efficacy may be performed using the detailed methods and insights described below, or by variations that are apparent to one of ordinary skill in the art. One approach to testing such inhibitors is the cell-based assay system described below. Other methods may be utilized. No limitation is intended with respect to how an Fst inhibitor is tested for therapeutic efficacy.

Polyclonal Antibodies (Abs). Polyclonal antibodies are prepared by immunizing an animal, such as a mouse, rat, hamster, guinea pig, rabbit, goat, sheep, chicken, or horse with a specific polypeptide or peptide fragments of Fst. Routes of administration for the immunization may include, but are not limited to intravenous, intraperitoneal, subcutaneous, intramuscular, intradermal, footpad, intranodal, or intrasplenic. To increase antigenicity, the peptide fragments might be linked to a carrier protein such as albumin or keyhole limpet hemocyanin. In a preferred embodiment, 30 ug of antigenic peptide fragment are used for immunization. After one or more immunizations of the recipient animal, sera is obtained and tested for the presence of antibodies to human follistatin using an enzyme-linked immunosorbent assay (ELISA). Antibodies which bind to follistatin may be used directly or, more preferably, purified to enhance utility using the cognate peptide immobilized on argaose-based resins. See, for example, Milstein Nature 266 (1977) 550; Kohler & Milstein Nature 256 (1975) 495; Antibodies A Lab Manual 1989; Rasmussen Biotechnol Lett 29 (2007) 845; Delahaut Methods 116 (2017) 4-11; Hanly ILAR 27 (1995) 93; Newcombe C, Newcombe A R J Chromatogr B Analyt Technol Biomed Life Sci 848 (2007) 2; Murphy Antibody Tech J 6 (2016)

Polyclonal Abs are then tested for potential therapeutic efficacy as discussed below. The animal's blood is collected, and a variety of techniques such as an enzyme-linked immunosorbent assay (ELISA), a radioimmunoassay (MA), an immunohistochemical staining, etc. can be used to measure the polyclonal antibody's titer in antiserum. Polyclonal antibodies can be purified from the complex mixtures in the serum using chromatographic or non-chromatographic techniques. Using chromatography-based methods, antibodies can be separated by passing them through a solid phase (eg, silica resin or beads, monolithic columns, or cellulose membranes) and allowing the antibodies to bind or pass through depending on which chromatographic methods are being utilized. These methodologies include different separation techniques, such as affinity-tag binding, ion-exchange, size-exclusion chromatography, or immunoaffinity chromatography. Using non-chromatography-based approaches, precipitation, flocculation, crystallization, filtration, aqueous two-phase partitioning techniques, and any combination thereof can be employed.

Anti-Fst Monoclonal Abs. Monoclonal antibodies (mAbs) are prepared using standard methodologies. Briefly, animals are immunized as given above for polyclonal Ab preparation. After verification that the immunized animal is producing relevant antibodies according to the assays described above lymphocytes are harvested from the Ab-producing animal (such as a mouse) and fused with myeloma cells according to the method of Kohler and Milstein (1975). See also Kunert Appl Microbiol Biotechnol 100 (2016) 3451; Roque Biotechnol Prog 20 (2004) 639; Maynard Annu Rev Bio Eng 2 (2000) 339.

Clones producing individual mAbs are then tested using the assay methods described below for therapeutic efficacy.

Bi-specific Antibodies that target both Fst and an Fst-binding protein such as myostatin (mst), activin, or bone morphogenetic protein (bmp) may be prepared. The method of Roque Biotechnol Prog 20 (2004) 639, is instructive.

Humanized antibodies. It is preferable to humanize the mAbs prepared by any of the above-referenced approaches (or by another appropriate method) by replacement of their constant regions with the Fc domains of human antibodies. Such a replacement has been shown to generate more clinically useful Abs with a lower likelihood of inducing side effects such as the development of neutralizing Abs in the recipient which may render the therapeutic mAb less effective or ineffective. Humanization of mAbs is well-described. See, for example, Roque Biotechnol Prog 20 (2004) 639, Kipriyanov Mol Biotech 26 (2004) 39, and Maynard Annu Rev Bio Eng 2 (2000) 339.

Often starting with monoclonal antibodies from rodent origin, the DNA segments encoding the rodent's variable regions that are specific for the target antigen are joined to the segments of DNA encoding a human constant region. By exchanging the variable regions of the human antibody heavy and light chain genes for those derived from the rodent monoclonal, the resulting chimeric (humanized) antibodies are 60-70% human. For murine monoclonal antibodies, the epitope or antigenic determinant region is contained only in the complementarity determining regions. Each domain, the heavy and light chains, have three of these regions surrounded by framework regions. To construct a monoclonal antibody that is 90-95% recognized as human, the complementarity determining regions of the murine monoclonal that were selected for a desired antigen can be adjoined to human framework regions.

A monoclonal antibody that is essentially 100% human can be obtained by genetically engineering the immune system of an animal, often a mouse using standard procedures.

Synthetic Abs. Synthetic antibodies are another approach to antibody creation. Such antibodies are created from synthetic libraries and may also be utilized to generate fully humanized, high affinity, high specificity antibodies for therapeutic use. The approach is analogous to the methods described above in terms of Ab functional activity See Shim BMB Reports 48 (2015) 489, Bradbury Nat Biotech 29 (2011) 245.

FAb′ fragments are prepared against Fst from anti-Fst mAbs according to standard methods. In addition, antigen binding fragments/F(ab) fragments, Variable fragments (Fv fragments), Single chain variable fragments (scFv fragments), and the like may also be prepared according to the methods of Hust BMC Biotech 7 (2007); Skerra Curr Opin Immunol 5 (1993) 256; Roque Biotechnol Prog 20 (2004) 639; Skerra-Pluckthun Science 240 (1988) 1038; Kipriyanov Mol Biotech 26 (2004) 39.

Antibody fragments are often produced in bacterial systems since they are small in size and can be produced in large quantities while maintaining function. Antigen binding fragments/F(ab) fragments may be prepared in recombinant systems as well. Variable fragments include both the heavy and light chains of the variable region on the antibody fragment that contain the antigen binding site. The antibodies or fragments are often expressed in the same bacterial cell, e.g., E. coli, and are secreted together into the periplasm of the bacteria. Using approximately equivalent amounts of each of the chains and secreting them essentially at the same time allows proper folding and assembly of a functional antibody fragment. Eukaryotic systems, such as yeast, insect, and mammalian cells, are also viable systems for the production of variable antibody (Fv) fragments.

For single chain variable fragments, the variable part of heavy chain and the variable part of the light chain of the antibody fragment that contain the antigen binding site of the whole antibody connected by a peptide linker are expressed in the same bacterial cell, such as an E. coli, and are secreted together into the periplasm of the bacteria.

Nanobodies. Nanobodies against Fst are prepared according to the methods as previously described. See, for example, Liu Mol Immunol 96 (2018) 37; Steeland Drug Discov Today 21 (July 2016) 1076; Angew Chem Int Ed Engl 57 (February 2018) 2314; Fridy Nat Methods 11 (2014) 1253; and Goldman Front Immunol July 2017.

Nanobodies are commonly obtained from any of the following created libraries—immune libraries, naïve libraries, or semi-synthetic/synthetic libraries. For immune libraries, antigen specific heavy chain antibodies undergo affinity maturation following immunization of animals most commonly from the Camelidae family. mRNA is obtained from peripheral blood lymphocytes and cDNA is synthesized by reverse transcription. Nanobodies are selected by screening the library using established techniques such as phage display, cell surface display, mRNA/cDNA display, HTS DNA sequencing and mass spec identification, biotinylated nanobody screening, or a bacterial-two-hybrid system.

For naïve libraries, phage display and ribosome display are common techniques used to select nanobodies generated from the mRNA obtained and cDNA synthesized from peripheral bloo lymphocytes collected from non-immunized animals.

For the semi-synthetic/synthetic libraries, the complementarity-determining regions of the nanobody are randomly changed in length and by sequence, while the framework regions are conserved. This allows for expansion of the library as well as for the generation of diversity within it.

Small molecule inhibitors of Fst. Compounds that (i) inhibit Fst binding to one or more of its binding proteins, including MST, BMP, or Activin, (ii) inhibit expression of a Fst gene, or (iii) enhance degradation of Fst may be identified using standard in-vitro cell-free radioligand or fluorescent-ligand binding assays, or their equivalent. The sources for small molecule inhibitors include, but are not limited to, for instance, chemical compound libraries, fermentation media of Streptomycetes, other bacteria and fungi, and cell extracts of plants and other vegetations. Small molecule libraries are available, and include AMRI library, AnalytiCon, BioFocus DPI Library, Chem-XInfinity, ChemBridge Library, ChemDiv Library, Enamine Library, The Greenpharma Natural Compound Library, Life Chemicals Library, LOPAC1280™, MicroSource Spectrum Collection, Pharmakon, The Prestwick Chemical Library®, SPECS, NIH Clinical Collection, Chiral Centers Diversity Library.

Gene Silencing using short interfering RNA (siRNA) and related approaches using RNA interference (RNAi). It is now well-established that RNAi play an important role in post-transcriptional gene silencing through molecules such as siRNAs. siRNAs are ˜19-22 nucleotide (nt) duplex RNA (dsRNA) molecules capable of reducing or silencing the translation of messenger RNAs (mRNAs) in a sequence specific fashion. See Walton et al., 2010; Sibley et al., 2010.

Polynucleotides can be used to reduce expression of specific genes. Such inhibitory polynucleotides include RNA interference (RNAi), mediated by double-stranded small interfering RNA (siRNA), which silences a gene with a high degree of specificity. A siRNA includes a sequence that is complementary to a protein coding messenger RNA (mRNA) and causes the degradation of the mRNA. One of ordinary skill in the art can design and synthesize siRNA molecules that are able to inhibit follistatin, as shown in the example given by Gao et al (2010). siRNA molecules for inhibition of follistatin are also commercially available (e.g., Dharmacon, Lafayette, Colo.). Automated synthesis of nucleic acids is well established, and includes modifications at numerous positions on the nucleoside and ribose/deoxyribose ring systems (Sibley et al., 2010; Walton et al., 2010).

Another type of inhibitory nucleotide includes antisense RNA, single stranded RNA complementary to a protein coding mRNA with which it hybridizes, and thereby blocks its translation into protein. A siRNA used in the methods herein has the ability to reduce expression of Fst315. RNA interference methods represent a useful approach for molecularly targeted therapy. Thus, in another embodiment, siRNAs or another RNAi methodology is utilized. siRNAs are synthesized and tested for their ability to reduce circulating Fst in a therapeutically effective manner in a mammal. Oligonucleotide synthetic methods of manufacturing siRNAs are well established. No limitation is intended with respect to the type of RNAi that may be utilized to reduce Fst levels in a mammal, including RNA-DNA chimeras, tandem hairpin RNAs, tandem siRNAs, tRNA-shRNAs, and the like (Sibley Mol Ther 18 (2010) 466).

In one embodiment, a polynucleotide useful herein include a double stranded RNA (dsRNA) polynucleotide. The sequence of a polynucleotide includes one strand, referred to herein as the sense strand, of 16 to 30 nucleotides, for instance, 16, 17, 18, 19, 20, 21, 22, 23, 24, 25, 26, 27, 28, 29, or 30 nucleotides. The sense strand is substantially identical, preferably, identical, to a target mRNA, e.g., an mRNA that encodes Fst315. As used herein, the term “identical” means the nucleotide sequence of the sense strand has the same nucleotide sequence as a portion of the target mRNA. As used herein, the term “substantially identical” means the sequence of the sense strand differs from the sequence of a target mRNA at 1, 2, or 3 nucleotides, preferably 1 nucleotide, and the remaining nucleotides are identical to the sequence of the mRNA. These 1, 2, or 3 nucleotides of the sense strand are referred to as non-complementary nucleotides. When a polynucleotide includes a sense strand that is substantially identical to a target mRNA, the 1, 2, or 3 non-complementary nucleotides are preferably located in the middle of the sense strand. For instance, if the sense strand is 21 nucleotides in length, the non-complementary nucleotides are typically at nucleotides 9, 10, 11, or 12, preferably nucleotides 10 or 11. The other strand of a dsRNA polynucleotide, referred to herein as the anti-sense strand, is complementary to the sense strand.

The sense and anti-sense strands of a dsRNA polynucleotide may also be covalently attached, typically by a spacer made up of nucleotides. Such a polynucleotide is often referred to in the art as a short hairpin RNA (shRNA). Upon base pairing of the sense and anti-sense strands, the spacer region forms a loop. The number of nucleotides making up the loop can vary, and loops between 3 and 23 nucleotides have been reported (Sui et al., Proc. Nat'l. Acad. Sci. USA, 99, 5515-5520 (2002), and Jacque et al., Nature, 418, 435-438 (2002)).

In one embodiment, a polynucleotide useful herein includes single stranded RNA (ssRNA) polynucleotides. The sequence of a polynucleotide includes one strand, referred to herein as the anti-sense strand, of at least 16 nucleotides. The anti-sense strand is substantially complementary, preferably, complementary, to a target mRNA, e.g., an mRNA that encodes Fst315. In one embodiment, a polynucleotide for decreasing expression of a coding region in a cell includes substantially all of a coding region, or in some cases, an entire coding region. An antisense strand is substantially complementary, preferably, complementary, to a target coding region or a target mRNA. As used herein, the term “substantially complementary” means that at least 1, 2, or 3 of the nucleotides of the antisense strand are not complementary to a nucleotide sequence of a target mRNA.

Polynucleotides of the present disclosure are preferably biologically active. A biologically active polynucleotide causes the post-transcriptional inhibition of expression, also referred to as silencing, of a target coding region. Without intending to be limited by theory, after introduction into a cell a polynucleotide of the present invention will hybridize with a target mRNA and signal cellular endonucleases to cleave the target mRNA. The result is the inhibition of expression of the polypeptide encoded by the mRNA. Whether the expression of a target coding region is inhibited can be determined by, for instance, measuring a decrease in the amount of the target mRNA in the cell, measuring a decrease in the amount of polypeptide encoded by the mRNA, or by measuring a decrease in the activity of the polypeptide encoded by the mRNA.

A polynucleotide of the present disclosure may include additional nucleotides. For instance, with respect to the sense strand, the 5′ end, the 3′ end, or both ends can include additional nucleotides, provided the additional nucleotides are identical to the appropriate target mRNA and the overall length of the sense strand is not greater than 30 nucleotides.

A polynucleotide may be modified. Such modifications can be useful to increase stability of the polynucleotide in certain environments. Modifications can include a nucleic acid sugar, base, or backbone, or any combination thereof. The modifications can be synthetic, naturally occurring, or non-naturally occurring. A polynucleotide can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide-nucleic acids. Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides. Polynucleotides can be obtained commercially synthesized to include such modifications (for instance, Dharmacon Inc., Lafayette, Colo.).

In one embodiment it is preferable to target compounds of the invention to the liver of the human being or other organism for which treatment with an Fst inhibitor is desired. As used herein, to “target” means to chemically modify a compound for the purpose of increasing the amount of the compound that enters the liver rather than other organs in the body. This is because Fst is produced in the liver of a mammal. Therefore, targeting a therapeutic compound to the liver will increase the efficacy of the compound for inhibition of follistatin.

Methods of targeting a compound to hepatocytes (a specific type of liver cell) are well known in the literature, and include addition of a targeting agent to a compound described herein, such as a polynucleotide, including a siRNA. One approach is to chemically conjugate targeting agent to a compound to a compound. An example of a targeting agent is an N-acetylgalactosamine (GalNAc) moiety (Nair et. al., 2014; Rajeev et al, 2015; Matsuda et. al., 2015). In one embodiment, a GalNAc moiety is conjugated to a nucleic acid sequence, such as an anti-sense oligonucleotide or an siRNA (Lee and Sinko, 2006; Willoughby et al. 2018). Since an asialoglycoprotein receptor (ASGPR) is expressed specifically on hepatocytes, and because GalNAc is a known ligand for the ASGPR, addition of a GalNAc moiety to a compound such as an siRNA results in a GalNAc-siRNA conjugate molecule that is rapidly cleared from the blood through binding to the ASGPR followed by subsequent internalization of the complex into clathrin-coated endosomes (Springer and Dowdy, 2018). In one embodiment, one or more

GalNAc moiety is conjugated to the 5′ end of the sense strand of the siRNA (Kumar et al., 2019; Willoughby et al. 2018, Wang et. al., 2017).

Other targeting agents are known that are capable of targeting compounds to receptors that are expressed in a tissue-specific manner such as on hepatocytes (in the liver), glial cells (nerves), adipocytes (fat), myocytes (muscle), and the like (Lee et. al., 2012). No limitation is intended on the nature of the targeting approach that may be utilized. For the purposes of this invention directed toward the inhibition of follistatin, targeting cells in the liver, and particularly hepatocytes, is preferable.

A polynucleotide useful in a method described herein can be administered directly to a patient. In those embodiments where the polynucleotide includes RNA, the RNA can be supplied indirectly by introducing a vector that encodes the RNA. For instance, when siRNA is the desired polynucleotide, the siRNA can be supplied indirectly by administering one or more vectors that encode both single strands of a dsRNA.

Gene Therapy. In another embodiment, viral vector-based gene therapy approaches may be utilized to reduce Fst expression or production in a mammal. No limitation is intended with respect to the type of gene therapy approach that may be utilized. In one preferred embodiment which has shown clinical efficacy with other targets, a viral vector system may be utilized to introduce anti-sense nucleic acids into Fst-producing organs such as the liver. Such methods are well-known in the art. In one preferred embodiment, Adeno-Associated Viruses (AAV) are utilized. One or more coding or non-coding anti-sense segments encoding a Fst protein are utilized in an AAV vector system for introduction into the liver of an afflicted mammal. See, for example, Naso BioDrugs 31 (2017) 317; Ojala Neuroscientist 21 (2015) 84; Hanna Health Policy 122 (2018) 217; Mendell NEJM 377 (2017) 1713.

Genome Editing. The Crispr/cas9 system as well as other genomic editing techniques may be utilized to endogenously modify cells in the liver or other tissue to reduce the expression of Fst. Reduced expression of Fst will result in lower levels of Fst protein and a concomitant improvement in insulin sensitivity in the periphery. See for example, Franco-Tormo et al., 2018; Li et al., 2018; and the standard methods disclosed therein.

Fst-binding polypeptides. Using standard approaches, peptide fragments selected from Fst, or alternatively from one of follistatin's known binding partners such as myostatin, bone morphogenetic protein, activin, etc. (see above) may be used to generate a peptide capable of blocking the interaction between Fst and a known binding partner. Soluble binding assays using radioligands, ELISA techniques, or fluorescently tagged ligands or antibodies are well known in the art. See, for example, (Horowitz, A. D. et al., 1981; Knudsen, L. et al., 2012)

No limitation is intended on the method by which a particular Fst binding compound is identified or enriched.

### Testing for Potential Therapeutic Efficacy: Cell Based Assay for Identifying Effective Fst Binding Compounds

A potential Fst inhibitor compound(s) is/are prepared from one or more of the methods described above and then tested for the ability to restore insulin signaling in an isolated animal or human adipocyte or 3T3L1 adipocyte, or isolated human or animal hepatocytes. The animal or cell is incubated with serum from LDKO-mice or another comparable source of Fst by assaying the relative increase in binding of PI3K to IRS1 under insulin stimulation. 3T3-L1 adipocytes are preferred for use in this cell-based assay as previously shown (Tao, R. et al., 2018). The formation and concentration of IRS1•p110 complex is quantified using an XMAP® binding assay on the Luminex™ platform.

3T3-L1 pre-adipocytes obtained from a mycoplasma-free stock are cultured in DMEM/F12 with 10% BCS in 5% CO₂. Two days post-confluence, cells are exposed to DMEM/10% FBS with isobutylmethylxanthine (0.5 mM), dexamethasone (1 μM) and insulin (5 μg/ml). After 2 days, cells are maintained in DMEM/10% FBS until ready for treatment at day 7. On day 9, cells are treated with insulin (10 nM) for 3 min after being maintained in DMEM/5% mouse serum from insulin resistance mice for 24 hours. Mouse serum from insulin resistant mice is useful as it provides a source of Fst and Fst targets that contribute to WAT insulin resistance (Tao, R. et al., 2018).

As described previously (Hancer et. al., 2014; Copps et. al., 2016), the IRS1 capture antibody (rabbit monoclonal antibody 58-10C-31, Millipore catalog number 05-784R) is coupled to magnetic carboxylated microspheres. The p110 subunit of PI3K associated with captured IRS1 is detected with antibodies from Cell Signaling Technology (CST #4249). For Luminex™ assays, cell lysates (10 μg) or mouse tissue lysates (80 μg) are diluted with Irs1 capture beads (4000 beads/well) in a total volume of 50 μl of phosphoprotein detection wash buffer (Bio-rad) and incubated overnight in 96-well round bottom plates. After washing twice with the same buffer, the beads are incubated with 50 μl of detection antibody for 1 h on a rotary plate shaker (80 rpm). After removal of the biotinylated detection antibody, the beads can be incubated with shaking in 25 μl of 1 μg/ml streptavidin-phycoerythrin (Prozyme) for 15 min. All solutions are then removed, and beads are suspended in PBS-BN (Sigma®) for analysis in a Luminex™ FlexMap 3D instrument.

Alternatively, another assay for identifying potentially therapeutic mAbs is to measure the degree of AKT phosphorylation following insulin stimulation in the presence or absence of selected anti-Fst Abs using cells exposed to serum from insulin-resistant LDKO mice. Tissue or 3T3-L1 adipocytes incubated with serum from insulin resistant LDKO-mice are homogenized in the lysis buffer (50 mm Hepes, pH 7.5, 150 mm NaCl, 10% glycerol, 1% Triton X-100, 1.5 mm MgCl₂, 1 mm EGTA, 10 mm sodium pyrophosphate, 100 mm sodium fluoride, and freshly added protease inhibitor cocktail and phosphatase inhibitor cocktail). Protein extracts are resolved on an SDS-PAGE gel and transferred to nitrocellulose membrane (Bio-Rad®). Detection of proteins is carried out by incubations with HRP-conjugated secondary antibodies targeted against regulatory phosphorylation sites in AKT—including T308 or S473—followed by ECL detection reagents.

The skilled person may design other assay systems that measure increases in insulin signaling of anti-follistatin Abs or other Fst inhibitor compounds under the conditions given above—including the use of an XMAP® assay to quantify AKT phosphorylation. Moreover, other downstream targets can be selected—including reduced HSL phosphorylation; reduced FOXO1 phosphorylation; increased S6K phosphorylation; or increased RPS6 phosphorylation No limitation is intended on how the compounds of the disclosure may be characterized for their ability to enhance these and other insulin signaling responses in an assay that measures insulin signaling and its release from inhibitory resistance owing to Fst.

For example, insulin normally promotes dephosphorylation of HSL in 3T3-L1 adipocytes. In this assay 3T3-L1 adipocytes incubated with 5% serum from insulin resistant LDKO-mice are stimulated with insulin for a few minutes. The cells are homogenized in the lysis buffer (50 mm Hepes, pH 7.5, 150 mm NaCl, 10% glycerol, 1% Triton X-100, 1.5 mm MgCl₂, 1 mm EGTA, 10 mm sodium pyrophosphate, 100 mm sodium fluoride, and freshly added protease inhibitor cocktail and phosphatase inhibitor cocktail). Protein extracts are resolved on an SDS-PAGE gel and transferred to nitrocellulose membrane (Bio-Rad). Detection of phosphorylated HSL at pS660^{Hsl} using phospho-HSL (Ser660) (Antibody #4126, Cell Signaling Technology) is carried out by incubations with HRP-conjugated secondary antibodies targeted against antibodies that bind to the regulatory phosphorylation sites in HSL—followed by ECL detection reagents.

Once effective mAbs or other effective compounds of the disclosure are identified in the aforementioned cellular assays, the compounds or Abs that score positively in the one of the assays given above may be further tested for in-vivo efficacy. Liver-specific Irs1 and Irs2 double knockout mice (LDKO) are preferably bred as previously described (27, 28). Alternatively, C57BL6 mice (Stock No. 000664), ob/ob mice (Stock No. 000632), B6.129S2-I16tm1Kopf/J mice (Stock No. 002650) can be purchased from The Jackson Lab (Bar Harbor, Me.). These mice are placed on the high fat diet to induce insulin resistance and diabetes between 4-16 weeks of age. Preferably, all mice are housed in plastic cages on a 12:12 h light-dark cycle with free access to water and food in an appropriate facility.

The hyperinsulinemic euglycemic clamp in conscious and unrestrained mice is used to assess the efficacy of the Fst inhibitors to inhibit Fst's ability to induce insulin resistance. Prior to the clamp experiment, one catheter is inserted into the right jugular vein for infusions. After 5-7 days of recovery, mice that lose less than 10% of their preoperative weight are subjected to the hyperinsulinemic euglycemic clamp.

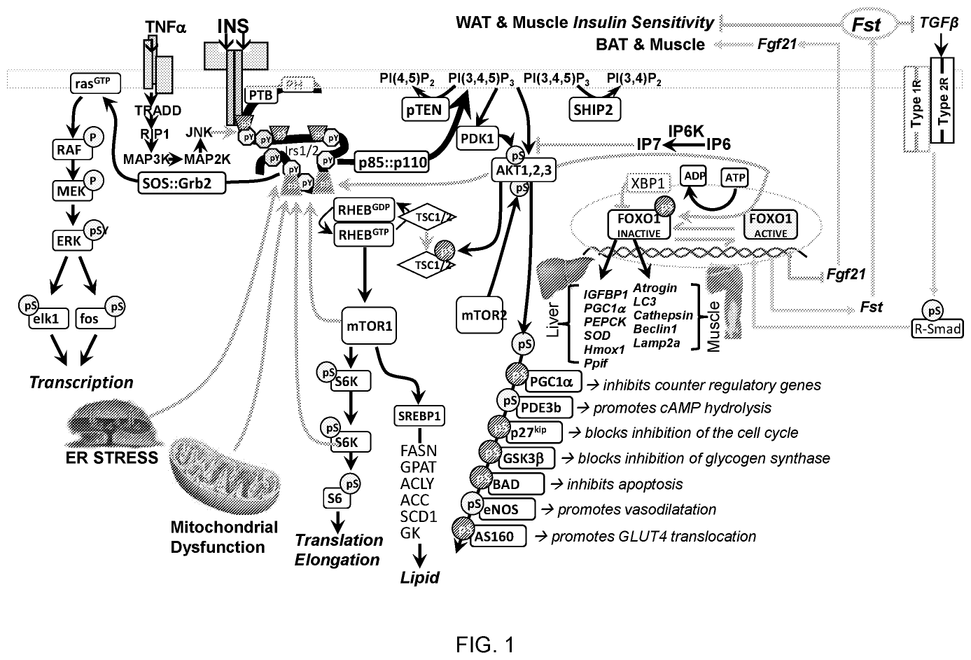

The day before the experiment the mice are treated with the Fst binding protein or antibody at concentrations determined in the cell-based assays of the previous section. On the day of the experiment, mice are deprived of food for 3.5 hours at 8:00 am and then infused continuously with D-[3-³H]-glucose (PerkinElmer®) (0.05 μCi/min) at a rate of 1 μl/min for 1.5 h. After basal sampling from the tail vein, a 140 min hyperinsulinemic euglycemic clamp is conducted with a primed-continuous infusion of human regular insulin (4 mU/kg/min, Humulin, Eli Lilly®) at a rate of 2 μl/min and continuously with D-[3-³H]-glucose (PerkinElmer®) (0.1 μCi/min) at a rate of 2 ul/min throughout the clamp experiment. The insulin solutions are prepared with 3% BSA in 0.9% saline. 20% glucose was infused at variable rates as needed to maintain plasma glucose at ˜130 mg/dl (except in FIG. 1D,F (Cntr±SEM: 138±9 mg/dl; LDKO±SEM: 204±21 mg/dl; P<0.05)). Preferably, all infusions are conducted with micro infusion pumps (KD Scientific or equivalent). Blood glucose concentrations are monitored regularly according to a fixed scheme from tail vein. To estimate insulin-stimulated glucose uptake in WAT, BAT and skeletal muscle, 2-deoxy-D-[1-¹⁴C] glucose (10 μCi/mice; PerkinElmer) is administered as a bolus at 95 min after the start of clamp. Blood samples (20 ul) are taken at −5, 100, 110, 120, 130, and 140 min of clamp for the measurement of plasma D-[3-³H]-glucose and 2-deoxy-D-[1-¹⁴C] glucose concentrations. Steady state is considered achieved during 100-140 min, when a fixed glucose-infusion rate maintains the glucose concentration in blood constantly for 40 min. At the end of the experiment, mice are sacrificed by ketamine/xylazine and WAT, BAT, skeletal muscle and liver are dissected and store at −80° C. for potential further analysis as necessary.

The D-[3-³H]-glucose and 2-deoxy-D-[1-¹⁴C] glucose concentrations in plasma are measured according to the procedure of “GLUCOSE CLAMPING THE CONSCIOUS MOUSE” from the Vanderbilt-NIDDK Mouse Metabolic Phenotyping Center with some modifications. Briefly, 6 μl of plasma sample mixed with 14 μl saline is treated with 100 ul 3N Ba(OH)₂ and ZnSO₄ (add Ba(OH)₂ prior to ZnSO₄) and 100 μl of supernatant is pipetted into a scintillation vial and dried in an oven overnight; 8 ml of scintillation fluid are added to the dried vial, or to 50 μl non-dry supernatant for measuring radioactivity in a liquid scintillation counter. For measuring 2-deoxy-D-[1-¹⁴C] glucose, lysates of adipose tissue and skeletal muscle are processed using a perchloric acid Ba(OH)₂/ZnSO₄ precipitation (Ferre, P. et al., 1985). Glucose uptake into WAT, BAT and skeletal muscle in vivo may be calculated based on 2-deoxy-D-[1-¹⁴C]-glucose 6-phosphate accumulation and specific activity of 2-deoxy-D-[1-¹⁴C]-glucose in serum.

Fst binding proteins or specific antibodies that promote insulin-suppression of hepatic glucose production are selected as biologically active candidates for the enhancement of insulin action by neuralizing the effect of Fst to promote insulin resistance.

### REFERENCES

Aguirre V, Uchida T, Yenush L, Davis R, White M F. The c-Jun NH(2)-terminal kinase promotes insulin resistance during association with insulin receptor substrate-1 and phosphorylation of Ser(307). J Biol Chem. 2000 Mar. 24; 275(12):9047-54.

Antibodies A Lab Manual 1989

Astrinidis A, Henske E P. Tuberous sclerosis complex: linking growth and energy signaling pathways with human disease. Oncogene 2005 Nov. 14; 24(50):7475-81.

Backer J M, Kahn C R, Cahill D A, Ullrich A, White M F. Receptor-mediated internalization of insulin requires a 12-amino acid sequence in the juxtamembrane region of the insulin receptor b-subunit. Journal of Biological Chemistry. 1990; 265:16450-4.

Barbieri M, Rizzo M R, Manzella D, Grella R, Ragno E, Carbonella M, Abbatecola A M, Paolisso G. Glucose regulation and oxidative stress in healthy centenarians. Exp Gerontol. 2003 January; 38(1-2):137-43.

Berge, S. M., Bighley, L. D., Monkhouse, D. C. Pharmaceutical salts. J Pharm Sci. 1977 January; 66(1):1-19.

Biddinger S B, Kahn C R. From mice to men: insights into the insulin resistance syndromes. Annu Rev Physiol. 2006; 68:123-58.

Bjornholm M, He A R, Attersand A, Lake S, Liu S C, Lienhard G E, Taylor S, Arner P, Zierath J R. Absence of functional insulin receptor substrate-3 (IRS-3) gene in humans. Diabetologia 2002 December; 45(12):1697-702.

Bradbury A R, Sidhu S, Dübel S, McCafferty J. Beyond natural antibodies: the power of in vitro display technologies. Nat Biotechnol. 2011 March; 29(3):245-54.

Brownlee M. The pathobiology of diabetic complications: a unifying mechanism. Diabetes 2005 June; 54 (6):1615-25.

Burks D J, Pons S, Towery H, SmithHall J, Myers M G, Yenush L, et al. Heterologous pleckstrin homology domains do not couple IRS-1 to the insulin receptor. Journal of Biological Chemistry. 1997; 272(44):27716-21.

Burks D J, Wang J, Towery H, Ishibashi O, Lowe D, Riedel H, et al. IRS pleckstrin homology domains bind to acidic motifs in proteins. Journal of Biological Chemistry. 1998; 273(47):31061-7.

Calnan D R, Brunet A. The FoxO code. Oncogene. 2008; 27(16):2276-88.

Campbell, I W. Metformin—life begins at 50: A symposium held on the occasion of the 43rd Annual Meeting of the European Association for the Study of Diabetes, Amsterdam, The Netherlands, September 2007, The British Journal of Diabetes & Vascular Disease. 2007 Sept. 7: 247-252

Chen L, Magliano D J, Zimmet P Z. The worldwide epidemiology of type 2 diabetes mellitus—present and future perspectives. Nature Reviews Endocrinology 2012, 8(4), 228-36.

Cheng Z, Guo S, Copps K, Dong X, Kollipara R, Rodgers J T, et al. Foxo1 integrates insulin signaling with mitochondrial function in the liver. Nat Med. 2009; 15(11):1307-11.

Cole G M, Frautschy S A. The role of insulin and neurotrophic factor signaling in brain aging and Alzheimer's Disease. Exp Gerontol 2007 January; 42(1-2):10-21.

Copps K D, Hançer N J, Qiu W, White M F. Serine 302 Phosphorylation of Mouse Insulin Receptor Substrate 1 (IRS1) Is Dispensable for Normal Insulin Signaling and Feedback Regulation by Hepatic S6 Kinase. J Biol Chem. 2016 Apr. 15; 291(16):8602-17.

Copps K D, White M F. Regulation of insulin sensitivity by serine/threonine phosphorylation of insulin receptor substrate proteins IRS1 and IRS2. Diabetologia. 2012; 55(10):2565-82.

D'Adamo E, Marcovecchio, M L, de Giorgis T, Chiavaroli V, Giannini C, Chiarelli F, Mohn A. Pediatric Nonalcoholic Fatty Liver Disease. Hot Topics in Endocrine and Endocrine-Related Diseases, May 8, 2013 Chapter 9.

DeFronzo R A, Tripathy D. Skeletal muscle insulin resistance is the primary defect in type 2 diabetes. Diabetes Care. 2009; 32 Suppl 2:S157-S63.

DeFronzo R A. Pathogenesis of type 2 diabetes mellitus. Med Clin North Am. 2004 July; 88(4):787-835, ix.

Delahaut P. Immunisation—Choice of host, adjuvants and boosting schedules with emphasis on polyclonal antibody production. Methods. 2017 Mar. 1; 116:4-11.

Dhe-Paganon S, Ottinger E A, Nolte R T, Eck M J, Shoelson S E. Crystal structure of the pleckstrin homology-phosphotyrosine binding (PH-PTB) targeting region of insulin receptor substrate 1. Proc Natl Acad Sci USA. 1999; 96(15):8378-83.

Diabetes Prevention Program Research Group. Reduction in the Incidence of Type 2 Diabetes with Lifestyle Intervention or Metformin. N Engl J Med 2002 Feb. 7; 346(6):393-403

Dong X, Park S, Lin X, Copps K, Yi X, White M F. Irs1 and Irs2 signaling is essential for hepatic glucose homeostasis and systemic growth. J Clin Invest. 2006 January; 116(1):101-14.

Dong X C, Copps K D, Guo S, Li Y, Kollipara R, DePinho R A, et al. Inactivation of hepatic Foxo1 by insulin signaling is required for adaptive nutrient homeostasis and endocrine growth regulation. Cell Metab. 2008; 8(1):65-76.

Eck M J, Dhe-Paganon S, Trub T, Nolte R T, Shoelson S E. Structure of the IRS-1 PTB domain bound to the juxtamembrane region of the insulin receptor. Cell. 1996; 85(5):695-705.

Farooq A, Plotnikova O, Zeng L, Zhou M M. Phosphotyrosine binding domains of Shc and insulin receptor substrate 1 recognize the NPXpY motif in a thermodynamically distinct manner. The Journal of biological chemistry. 1999; 274(10):6114-21.

Ferre P, Leturque A, Burnol A F, Penicaud L, Girard J. A method to quantify glucose utilization in vivo in skeletal muscle and white adipose tissue of the anaesthetized rat. Biochem J. 1985; 228(1):103-10.

Franco-Tormo M J, Salas-Crisostomo M, Rocha N B, Budde H, Machado S, Murillo-Rodríguez E. CRISPR/Cas9, the Powerful New Genome-Editing Tool for Putative Therapeutics in Obesity. J Mol Neurosci. 2018 May 7. doi: 10.1007/s12031-018-1076-4. [Epub ahead of print]

Fridy P C, Li Y, Keegan S, Thompson M K, Nudelman I, Scheid J F, Oeffinger M, Nussenzweig M C, Fenyö D, Chait B T, Rout M P. A robust pipeline for rapid production of versatile nanobody repertoires. Nat Methods. 2014 December; 11(12):1253-60.

Galfre G, Howe S C, Milstein C, Butcher G W, Howard J C. Antibodies to major histocompatibility antigens produced by hybrid cell lines. Nature. 1977 Apr. 7; 266(5602):550-2.

Gao X, Wei S, Lai K, Sheng J, Su J, Zhu J, Dong H, Hu H, Xu Z. Nucleolar follistatin promotes cancer cell survival under glucose-deprived conditions through inhibiting cellular rRNA synthesis. J Biol Chem. 2010 Nov. 19; 285 (47):36857-64.

George M M, Copeland K C. Current treatment options for type 2 diabetes mellitus in youth: today's realities and lessons from the TODAY study. Curr Diab Rep. 2013 February; 13(1):72-80.

Giraud J, Haas M, Feener E P, Copps K D, Dong X, Dunn S L, White M F. Phosphorylation of Irs1 at SER-522 inhibits insulin signaling. Mol Endocrinol. 2007 September; 21(9):2294-302.

Goldman E R, Liu J L, Zabetakis D, Anderson G P. Enhancing Stability of Camelid and Shark Single Domain Antibodies: An Overview. Front Immunol. 2017 Jul. 25; 8:865.

Grusch, 2010, Atlas Genet Cytogenet Oncol Haematol., 14(12):1132-1135

Guo S, Copps K D, Dong X, Park S, Cheng Z, Pocai A, Rossetti L, Sajan M, Farese R V, White M F. The Irs1 branch of the insulin signaling cascade plays a dominant role in hepatic nutrient homeostasis. Mol. and Cell Biology. 2009, 29, 18, 5070-83.

Hagiwara A, Cornu M, Cybulski N, Polak P, Betz C, Trapani F, Terracciano L, Heim M H, Rüegg M A, Hall M N. Hepatic mTORC2 Activates Glycolysis and Lipogenesis through Akt, Glucokinase, and SREBP1c. Cell Metab. 2012 May 2; 15(5):725-38.

Han H Q, Zhou X, Mitch W E, Goldberg A L. Myostatin/activin pathway antagonism: molecular basis and therapeutic potential. The international journal of biochemistry & cell biology. 2013; 45(10):2333-47.

Hancer N J, Qiu W, Cherella C, Li Y, Copps K D, White M F. Insulin and metabolic stress stimulate multisite serine/threonine phosphorylation of insulin receptor substrate 1 and inhibit tyrosine phosphorylation. Journal of Biological Chemistry. 2014; 289(18):12467-84.

Hanly W C, Artwohl J E, Bennett B T. Review of Polyclonal Antibody Production Procedures in Mammals and Poultry. ILAR J. 1995; 37(3):93-118.

Hanna E, Toumi M, Dussart C, Borissov B, Dabbous O, Badora K, Auquier P. Funding breakthrough therapies: A systematic review and recommendation. Health Policy. 2018 March; 122(3):217-229

Hansen J, Rinnov A, Krogh-Madsen R, Fischer C P, Andreasen A S, Berg R M, Moller K, Pedersen B K, Plomgaard P. Plasma follistatin is elevated in patients with type 2 diabetes: relationship to hyperglycemia, hyperinsulinemia, and systemic low-grade inflammation. Diabetes Metab Res Rev. 2013; 29(6):463-72.

Hansen J S, Plomgaard P. Circulating follistatin in relation to energy metabolism. Mol Cell Endocrinol. 2016 Sep. 15; 433:87-93. 27264073.

Herzig S, Liver: A Target of Late Diabetic Complications. Exp Clin Endocrinol Diabetes 2012, 120, 202-4.

Horowitz A D, Greenebaum E, Weinstein I B. Identification of receptors for phorbol ester tumor promoters in intact mammalian cells and of an inhibitor of receptor binding in biologic fluids. Proc Natl Acad Sci USA. 1981 April; 78(4):2315-9.

Hotamisligil G S. Inflammation and metabolic disorders. Nature 2006 Dec. 14; 444(7121):860-7.

Housey G M, Balash M. Plant Extracts with Anti-Diabetic and Other Useful Activities. PCT Application. WO 2014/165297, Filed Mar. 12, 2014.

Housey, G M, White, M F, Method of Screening Activators and/or Inhibitors of Insulin Receptor Substrate 2. U.S. Pat. No. 8,557,512 B2. Ser. No. 10/541,263. Filed Dec. 31, 2003. Issued Oct. 15, 2013

http://www.diabetes.org/diabetes-basics/statistics

Hu C, Pang S, Kong X, Velleca M, Lawrence J C, Jr. Molecular cloning and tissue distribution of PHAS-I, an intracellular target for insulin and growth factors. Proc Natl Acad Sci USA 1994; 91:3730-4.

Hubbard S R. Juxtamembrane autoinhibition in receptor tyrosine kinases. Nat Rev Mol Cell Biol. 2004; 5(6):464-71.

Hubbard S R. The insulin receptor: both a prototypical and atypical receptor tyrosine kinase. Cold Spring Harb Perspect Biol. 2013; 5(3):1-12.

Humphrey S J, Yang G, Yang P, Fazakerley D J, Stockli J, Yang J Y, et al. Dynamic adipocyte phosphoproteome reveals that Akt directly regulates mTORC2. Cell Metab. 2013; 17(6):1009-20.

Hust M, Jostock T, Menzel C, Voedisch B, Mohr A, Brenneis M, Kirsch M I, Meier D, Dübel S. Single chain Fab (scFab) fragment. BMC Biotechnol. 2007 Mar. 8; 7:14.

Karlsson H K, Zierath J R. Insulin signaling and glucose transport in insulin resistant human skeletal muscle. Cell Biochem Biophys. 2007; 48(2-3):103-13.

Kendall D L, Amin R, Clayton P E. Metformin in the treatment of obese children and adolescents at risk of type 2 diabetes. Paediatr Drugs. 2014 February; 16(1):13-20.

Kido Y, Burks D J, Withers D, Bruning J C, Kahn C R, White M F, et al. Tissue-specific insulin resistance in mice with mutations in the insulin receptor, IRS-1, and IRS-2. J Clin Invest. 2000; 105(2):199-205.

Kilpeläinen T O, Zillikens M C, Stanc̆ákova A, Finucane F M, Ried J S, Langenberg C, Zhang W, Beckmann J S, Luan J, Vandenput L, et. al. Genetic variation near IRS1 associates with reduced adiposity and an impaired metabolic profile. Nat Genet. 2011 Jun. 26; 43(8):753-60.

Kipriyanov S M, Le Gall F. Generation and production of engineered antibodies. Mol Biotechnol. 2004 January; 26(1):39-60.

Kitabchi A E, Nyenwe E A. Hyperglycemic crises in diabetes mellitus: diabetic ketoacidosis and hyperglycemic hyperosmolar state. Endocrinol Metab Clin North Am 2006 December; 35(4):725-51, viii.

Knudsen L, Hansen B F, Jensen P, Pedersen T Å, Vestergaard K, Schäffer L, Blagoev B, Oleksiewicz M B, Kiselyov V V, De Meyts P. Agonism and antagonism at the insulin receptor. PLoS One. 2012; 7(12):e51972.

Köhler G, Milstein C. Continuous cultures of fused cells secreting antibody of predefined specificity. Nature. 1975 Aug. 7; 256(5517):495-7.

Kubota N, Kubota T, Itoh S, Kumagai H, Kozono H, Takamoto I, Mineyama T, Ogata H, Tokuyama K, Ohsugi M, Sasako T, Moroi M, Sugi K, Kakuta S, Iwakura Y, Noda T, Ohnishi S, Nagai R, Tobe K, Terauchi Y, Ueki K, Kadowaki T. Dynamic functional relay between insulin receptor substrate 1 and 2 in hepatic insulin signaling during fasting and feeding. Cell Metab. 2008 July; 8(1):49-64.

Kubota N, Tobe K, Terauchi Y, Eto K, Yamauchi T, Suzuki R, Tsubamoto Y, Komeda K, Nakano R, Miki H, Satoh S, Sekihara H, Sciacchitano S, Lesniak M, Aizawa S, Nagai R, Kimura S, Akanuma Y, Taylor S I, Kadowaki T. Disruption of insulin receptor substrate 2 causes type 2 diabetes because of liver insulin resistance and lack of compensatory beta-cell hyperplasia. Diabetes 2000 November; 49(11):1880-9.

Kumar P, Parmar R G, Brown C R, Willoughby J L S, Foster D J, Babu I R, Schofield S, Jadhav V, Charisse K, Nair J K, Rajeev K G, Maier M A, Egli M, Manoharan M. 5′-Morpholino modification of the sense strand of an siRNA makes it a more effective passenger. Chem Commun (Camb). 2019 Apr. 25, 55(35):5139-5142.

Kunert R, Reinhart D. Advances in recombinant antibody manufacturing. Appl Microbiol Biotechnol. 2016 April; 100(8):3451-61.

Laplante M, Sabatini D M. An emerging role of mTOR in lipid biosynthesis. Curr Biol. 2009 Dec. 1; 19(22):R1046-R1052.

Lee S H, Sinko P J. siRNA-Getting the message out. Eur J Pharm Sci. 2006 Apr., 27(5):401-10.

Lee S-K, Siefert A, Beloor J, Fahmy T M, Kumar P. Chapter five—Cell-Specific siRNA Delivery by Peptides and Antibodies. Methods in Enzymology. Wittrup, K. D., Verdine, G. L., editors. Academic Press, 2012; 502; 91-122

Lemmon M A, Ferguson K M, Abrams C S. Pleckstrin homology domains and the cytoskeleton. FEBS Lett. 2002; 513(1):71-6.

Lemmon M A, Ferguson K M, Schlessinger J. PH domains: Diverse sequences with a common fold recruit signaling molecules to the cell surface. Cell. 1996; 85(5):621-4.

Li L, Hu S, Chen X. Non-viral delivery systems for CRISPR/Cas9-based genome editing: Challenges and opportunities. Biomaterials. 2018 July; 171:207-218. doi:10.1016/j.biomaterials.2018.04.031. Epub 2018 Apr. 18.

Lipman T H, Levitt Katz L E, Ratcliffe S J, Murphy K M, Aguilar A, Rezvani I, Howe C J, Fadia S, Suarez E. Increasing incidence of type 1 diabetes in youth: twenty years of the Philadelphia Pediatric Diabetes Registry. Diabetes Care. 2013 June; 36(6):1597-603.

Liu W, Song H, Chen Q, Yu J, Xian M, Nian R, Feng D. Recent advances in the selection and identification of antigen-specific nanobodies. Mol Immunol. 2018 April; 96:37-47. doi: 10.1016/j.molimm.2018.02.012. Review. Pub Med PMID: 29477934.

Manning B D, Cantley L C. AKT/PKB signaling: navigating downstream. Cell. 2007; 129(7):1261-74.

Matsuda S, Keiser K, Nair J K, Charisse K, Manoharan R M, Kretschmer P, Peng C G, V Kel'in A, Kandasamy P, Willoughby J L, Liebow A, Querbes W, Yucius K, Nguyen T, Milstein S, Maier M A, Rajeev K G, Manoharan M. siRNA conjugates carrying sequentially assembled trivalent N-acetylgalactosamine linked through nucleosides elicit robust gene silencing in vivo in hepatocytes. ACS Chem Biol. 2015 May 15, 10(5):1181-7.

Maynard J, Georgiou G. Antibody engineering. Annu Rev Biomed Eng. 2000; 2:339-76.

Mendell J R, Al-Zaidy S, Shell R, Arnold W D, Rodino-Klapac L R, Prior T W, Lowes L, Alfano L, Berry K, Church K, Kissel J T, Nagendran S, L'Italien J, Sproule D M, Wells C, Cardenas J A, Heitzer M D, Kaspar A, Corcoran S, Braun L, Likhite S, Miranda C, Meyer K, Foust K D, Burghes A H M, Kaspar B K. Single-Dose Gene-Replacement Therapy for Spinal Muscular Atrophy. N Engl J Med. 2017 Nov. 2; 377(18): 1713-1722.

Menke A, Casagrande S, Geiss L, Cowie C C. Prevalence of and Trends in Diabetes Among Adults in the United States, 1988-2012. JAMA. 2015 Sep. 8; 314(10):1021-9.

Menon S, Yecies J L, Zhang H H, Howell J J, Nicholatos J, Harputlugil E, Bronson R T, Kwiatkowski D J, Manning B D. Chronic Activation of mTOR Complex 1 Is Sufficient to Cause Hepatocellular Carcinoma in Mice. Sci Signal. 2012; 5(217):ra24.

Monzavi R, Dreimane D, Geffner M E, Braun S, Conrad B, Klier M, Kaufman F R. Improvement in risk factors for metabolic syndrome and insulin resistance in overweight youth who are treated with lifestyle intervention. Pediatrics 2006 June; 117(6):e1111-e1118.

Murphy C, Devine T, O'Kennedy R. Technology advancements in antibody purification. Antibody Technology Journal. 2016 Aug. 26; 6: 17-22.

Nair J K, Willoughby J L, Chan A, Charisse K, Alam M R, Wang Q, Hoekstra M, Kandasamy P, Kel'in A V, Milstein S, Taneja N, O'Shea J, Shaikh S, Zhang L, van der Sluis R J, Jung M E, Akinc A, Hutabarat R, Kuchimanchi S, Fitzgerald K, Zimmermann T, van Berkel T J, Maier M A, Rajeev K G, Manoharan M. Multivalent N-acetylgalactosamine-conjugated siRNA localizes in hepatocytes and elicits robust RNAi-mediated gene silencing. J Am Chem Soc. 2014 Dec. 10, 136(49):16958-61.

Nandi A, Kitamura T, Kahn C R, Accili D. Mouse models of insulin resistance. Physiol Rev. 2004 April; 84(2):623-47.

Naso M F, Tomkowicz B, Perry W L 3rd, Strohl W R. Adeno-Associated Virus (AAV) as a Vector for Gene Therapy. Bio Drugs. 2017 August; 31(4):317-334.

Newcombe C, Newcombe A R. Antibody production: polyclonal-derived biotherapeutics. J Chromatogr B Analyt Technol Biomed Life Sci. 2007 Mar. 15; 848(1):2-7.

Norquay L D, D'Aquino K E, Opare-Addo L M, Kuznetsova A, Haas M, Bluestone J A, White, M F. Insulin receptor substrate-2 in beta-cells decreases diabetes in nonobese diabetic mice. Endocrinology 2009 October; 150(10):4531-40.

Numan S, Russell D S. Discrete expression of insulin receptor substrate-4 mRNA in adult rat brain. Brain Res Mol Brain Res. 1999 Sep. 8; 72(1):97-102.

Obsil T, Obsilova V. Structure/function relationships underlying regulation of FOXO transcription factors. Oncogene. 2008; 27(16):2263-75.

Ojala D S, Amara D P, Schaffer D V. Adeno-associated virus vectors and neurological gene therapy. Neuroscientist. 2015 February; 21(1):84-98.

Ozcan U, Cao Q, Yilmaz E, Lee A H, Iwakoshi N N, Ozdelen E, Tuncman G, Görgün C, Glimcher L H, Hotamisligil G S. Endoplasmic reticulum stress links obesity, insulin action, and type 2 diabetes. Science 2004 Oct. 15; 306(5695):457-61.

Pelicci G L, Lanfrancone L, Grignani F, McGlade J, Cavallo F, Forni G, et al. A novel transforming protein (SHC) with an SH2 domain is implicated in mitogenic signal transduction. Cell. 1992; 70(1):93-104.

Perakakis N, Kokkinos A, Peradze N, Tentolouris N, Ghaly W, Tsilingiris D, Alexandrou A, Mantzoros C S. Follistatins in glucose regulation in healthy and obese individuals. Diabetes Obes Metab. 2019 March, 21(3):683-690.

Pervin S, Singh V, Tucker A, Collazo J, Singh R. Modulation of transforming growth factor-β/follistatin signaling and white adipose browning: therapeutic implications for obesity related disorders. Horm Mol Biol Clin Investig. 2017 Sep. 9; 31(2).

Petersen K F, Dufour S, Savage D B, Bilz S, Solomon G, Yonemitsu S, Cline G W, Befroy D, Zemany L, Kahn B B, Papademetris X, Rothman D L, Shulman G I. The role of skeletal muscle insulin resistance in the pathogenesis of the metabolic syndrome. Proc Natl Acad Sci USA 2007 Jul. 31; 104(31):12587-94.

Previs S F, Withers D J, Ren J M, White M F, Shulman G I. Contrasting effects of IRS-1 vs IRS-2 gene disruption on carbohydrate and lipid metabolism in vivo. J Biol Chem. 2000 Sep. 19; 275(50):38990-4.

Rajeev K G, Nair J K, Jayaraman M, Charisse K, Taneja N, O'Shea J, Willoughby J L, Yucius K, Nguyen T, Shulga-Morskaya S, Milstein S, Liebow A, Querbes W, Borodovsky A, Fitzgerald K, Maier M A, Manoharan M. Hepatocyte-specific delivery of siRNAs conjugated to novel non-nucleosidic trivalent N-acetylgalactosamine elicits robust gene silencing in vivo. Chembiochem. 2015 Apr. 13, 16(6):903-8.

Rasmussen S K, Rasmussen L K, Weilguny D, Tolstrup A B. Manufacture of recombinant polyclonal antibodies. Biotechnol Lett. 2007 June; 29(6):845-52.

Reaven G. The metabolic syndrome or the insulin resistance syndrome? Different names, different concepts, and different goals. Endocrinol Metab Clin North Am. 2004; 33(2):283-303.

Reaven G M. Pathophysiology of insulin resistance in human disease. Physiol Rev. 1995 July; 75(3):473-86.

Roque A C, Lowe C R, Taipa M A. Antibodies and genetically engineered related molecules: production and purification. Biotechnol Prog. 2004 May-June; 20(3):639-54.

Rung J, Cauchi S, Albrechtsen A, Shen L, Rocheleau G, Cavalcanti-Proença C, Bacot F, Balkau B, Belisle A, Borch-Johnsen K, et. al. Genetic Variant near IRS1 is associated with Type 2 diabetes, insulin resistance and hyperinsulinemia. Nat Genet. 2009 October; 41(10):1110-5.

Samaan M C. Management of pediatric and adolescent type 2 diabetes. Int J Pediatr. 2013; 972034.

Savoye M, Caprio S, Dziura J, Camp A, Germain G, Summers C, Li F, Shaw M, Nowicka P, Kursawe R, Depourcq F, Kim G, Tamborlane W V. Reversal of early abnormalities in glucose metabolism in obese youth: results of an intensive lifestyle randomized controlled trial. Diabetes Care. 2014 February; 37(2):317-24.

Sawka-Verhelle D, Baron V, Mothe I, Filloux C, White M F, Van Obberghen E. Tyr₆₂₄ and Tyr₆₂₈ in insulin receptor substrate-2 mediate its association with the insulin receptor. J Biol Chem. 1997 Jun. 27; 272(26): 16414-20.

Sawka-Verhelle D, Tartare-Deckert S, White M F, Van Obberghen E. Insulin receptor substrate-2 binds to the insulin receptor through its phosphotyrosine-binding domain and through a newly identified domain comprising amino acids 591-786. J Biol Chem. 1996 Mar. 15; 271(11):5980-3.

Schattenberg J M, Schuppan D. Nonalcoholic steatohepatitis: the therapeutic challenge of a global epidemic. Current Opinion in Lipidology 2011, 22 (6), 479-88.

Schneyer A L, Wang Q, Sidis Y, Sluss P M. Differential distribution of follistatin isoforms: application of a new FS315-specific immunoassay. J Clin Endocrinol Metab. 2004 October; 89(10):5067-75.

Schumacher D, Helma J, Schneider A F L, Leonhardt H, Hackenberger C P R. Nanobodies: Chemical Functionalization Strategies and Intracellular Applications. Angew Chem Int Ed Engl. 2018 Feb. 23; 57(9):2314-2333.

Semple R K, Sleigh A, Murgatroyd P R, Adams C A, Bluck L, Jackson S, Vottero A, Kanabar D, Charlton-Menys V, Durrington P, Soos M A, Carpenter T A, Lomas D J, Cochran E K, Gorden P, O'Rahilly S, Savage D B. Postreceptor insulin resistance contributes to human dyslipidemia and hepatic steatosis. J Clin Invest. 2009 February; 119(2):315-22.

Shim H. Synthetic approach to the generation of antibody diversity. BMB Rep. 2015 September; 48(9):489-94.

Shimasaki S, Koga M, Esch F, Cooksey K, Mercado M, Koba A, Ueno N, Ying S Y, Ling N, Guillemin R. Primary structure of the human follistatin precursor and its genomic organization. Proc Natl Acad Sci USA. 1988 June; 85(12):4218-22.

Shimomura I, Matsuda M, Hammer R E, Bashmakov Y, Brown M S, Goldstein J L. Decreased IRS-2 and increased SREBP-1c lead to mixed insulin resistance and sensitivity in livers of lipodystrophic and ob/ob mice. Mol Cell. 2000 July; 6(1):77-86.

Sibley C R, Seow Y, Wood M J. Novel RNA-based strategies for therapeutic gene silencing. Mol Ther. 2010 March; 18(3):466-76.

Singh R, Braga M, Pervin S. Regulation of brown adipocyte metabolism by myostatin/follistatin signaling. Front Cell Dev Biol. 2014 Oct. 16; 2:60.

Skerra A, Plückthun A. Assembly of a functional immunoglobulin Fv fragment in Escherichia coli. Science. 1988 May 20; 240(4855):1038-41.

Skerra A. Bacterial expression of immunoglobulin fragments. Curr Opin Immunol. 1993 April; 5(2):256-62.

Springer A D, Dowdy S F. GalNAc-siRNA Conjugates: Leading the Way for Delivery of RNAi Therapeutics. Nucleic Acid Ther. 2018 June; 28(3):109-118.

Statistics from ADA http://www.diabetes.org/diabetes-basics/diabetes-statistics/

Steeland S, Vandenbroucke R E, Libert C. Nanobodies as therapeutics: big opportunities for small antibodies. Drug Discov Today. 2016 July; 21(7):1076-113.

Stentz F B, Umpierrez G E, Cuervo R, Kitabchi A E. Proinflammatory cytokines, markers of cardiovascular risks, oxidative stress, and lipid peroxidation in patients with hyperglycemic crises. Diabetes 2004 August; 53(8):2079-86.

Stumvoll M, Goldstein B J, van Haeften T W. Type 2 diabetes: principles of pathogenesis and therapy. Lancet 2005 Apr. 9; 365(9467):1333-46.

Sugino K, Kurosawa N, Nakamura T, Takio K, Shimasaki S, Ling N, Titani K, Sugino H. Molecular heterogeneity of follistatin, an activin-binding protein. Higher affinity of the carboxyl-terminal truncated forms for heparan sulfate proteoglycans on the ovarian granulosa cell. J Biol Chem. 1993 Jul. 25; 268(21):15579-87.

Taniguchi C M, Emanuelli B, Kahn C R. Critical nodes in signalling pathways: insights into insulin action. Nat Rev Mol Cell Biol. 2006 February; 7(2):85-96.

Taniguchi C M, Ueki K, Kahn C R. Complementary roles of IRS-1 and IRS-2 in the hepatic regulation of metabolism. J Clin Invest. 2005 March; 115(3):718-27.

Tao R, Wang C, Stöhr O, Qiu W, Hu Y, Miao J, Dong X C, Leng S, Stefater M, Stylopoulos N, Lin L, Copps K D, White M F. Inactivating hepatic follistatin alleviates hyperglycemia. Nature Medicine 2018 July, 24(7):1058-1069.Terauchi Y, Takamoto I, Kubota N, Matsui J, Suzuki R, Komeda K, Hara A, Toyoda Y, Miwa I, Aizawa S, Tsutsumi S, Tsubamoto Y, Hashimoto S, Eto K, Nakamura A, Noda M, Tobe K, Aburatani H, Nagai R, Kadowaki T. Glucokinase and IRS-2 are required for compensatory beta cell hyperplasia in response to high-fat diet-induced insulin resistance. J Clin Invest. 2007 January; 117(1):246-57.

Tsunekawa S, Demozay D, Briaud I, McCuaig, J, Accili D, Stein R, Rhodes, C J. 1FoxO Feedback Control of Basal IRS-2 Expression in Pancreatic b-Cells Is Distinct From That in Hepatocytes. Diabetes, 2011, (60), 2886-91.

Tuomilehto J. The emerging global epidemic of type 1 diabetes. Curr Diab Rep. 2013 December; 13(6):795-804.

Ungerleider N A, Bonomi L M, Brown M L, Schneyer A L. Increased activin bioavailability enhances hepatic insulin sensitivity while inducing hepatic steatosis in male mice. Endocrinology. 2013; 154(6):2025-33.

van der Horst A, Burgering B M. Stressing the role of FoxO proteins in lifespan and disease. Nat Rev Mol Cell Biol. 2007; 8(6):440-50.

Vanhaesebroeck B, Stephens L, Hawkins P. PI3K signalling: the path to discovery and understanding. Nat Rev Mol Cell Biol. 2012; 13(3):195-203.

Vaxillaire M, Veslot J, Dina C, Proenca C, Cauchi S, Charpentier G, Tichet J, Fumeron F, Mane M, Meyre D, Balkau B, Froguel P. DESIR Study Group. Impact of common type 2 diabetes risk polymorphisms in the DESIR prospective study. Diabetes 2008 January; 57(1):244-54.

Walton S P, Wu M, Gredell J A, Chan C. Designing highly active siRNAs for therapeutic applications. FEBS J. 2010 December; 277(23):4806-13.

Wan M, Leavens K F, Saleh D, Easton R M, Guertin D A, Peterson T R, Kaestner K H, Sabatini D M, Birnbaum M J. Postprandial Hepatic Lipid Metabolism Requires Signaling through Akt2 Independent of the Transcription Factors FoxA2, FoxO1, and SREBP1c. Cell Metab. 2011 Oct. 5; 14(4):516-27.

Wang N, Tall A R. A New Approach to PCSK9 Therapeutics. Circ Res. 2017 Mar. 31, 20(7):1063-1065. Wellen K E, Hotamisligil G S. Inflammation, stress, and diabetes. J Clin Invest. 2005 May; 115(5):1111-9.

White M F, Copps K D, Ozcan U, Tseng Y D. The Molecular Basis of insulin action. In: Jameson J L, DeGroot L J, editors. Endocrinology. 6th ed. Philadelphia: Elsevier; 2010. p. 636-59.

White M F, Livingston J N, Backer J M, Lauris V, Dull T J, Ullrich A, et al. Mutation of the insulin receptor at tyrosine 960 inhibits signal transmission but does not affect its tyrosine kinase activity. Cell. 1988; 54:641-9.

White M F. Insulin signaling in health and disease. Science 2003 Dec. 5; 302(5651):1710-1.

White M F. Regulating insulin signaling and beta-cell function through IRS proteins. Can J Physiol Pharmacol. 2006 July; 84(7):725-37.

Willoughby J L S, Chan A, Sehgal A, Butler J S, Nair J K, Racie T, Shulga-Morskaya S, Nguyen T, Qian K, Yucius K, Charisse K, van Berkel T J C, Manoharan M, Rajeev K G, Maier M A, Jadhav V, Zimmermann T S. Evaluation of GalNAc-siRNA Conjugate Activity in Pre-clinical Animal Models with Reduced Asialoglycoprotein Receptor Expression. Mol Ther. 2018 Jan. 3, 26(1):105-114.

Withers D J, Gutierrez J S, Towery H, Burks D J, Ren J M, Previs S, Zhang Y, Bernal D, Pons S, Shulman G I, Bonner-Weir S, White M F. Disruption of IRS-2 causes type 2 diabetes in mice. Nature 1998; 391(6670):900-4.

Wu J, Tseng Y D, Xu C F, Neubert T A, White M F, Hubbard S R. Structural and biochemical characterization of the KRLB region in insulin receptor substrate-2. Nature structural & molecular biology. 2008; 15(3):251-8.

Yecies J L, Zhang H H, Menon S, Liu S, Yecies D, Lipovsky A I, Gorgun C, Kwiatkowski D J, Hotamisligil G S, Lee C H, Manning B D. Akt stimulates hepatic SREBP1c and lipogenesis through parallel mTORC1-dependent and independent pathways. Cell Metab. 2011 Jul. 6; 14(1):21-32.

Yenush L, Makati K J, SmithHall J, Ishibashi O, Myers M G, White M F. The pleckstrin homology domain is the principle link between insulin receptor and IRS-1. Journal of Biological Chemistry. 1996; 271(39):24300-6.

Yisahak S F, Beagley J, Hambleton I R, Narayan K M. Diabetes in North America and The Caribbean: An update. Diabetes Res Clin Pract. 2014 February; 103(2):223-30.

Zhang L, Liu K, Han B, Xu Z, Gao X. The emerging role of follistatin under stresses and its implications in diseases. Gene. 2018 Jan. 10; 639:111-116.

Zhao C, Qiao C, Tang R H, Jiang J, Li J, Martin C B, Bulaklak K, Li J, Wang D W, Xiao X. Overcoming Insulin Insufficiency by Forced Follistatin Expression in beta-cells of db/db Mice. Molecular therapy: the journal of the American Society of Gene Therapy. 2015; 23(5):866-74.

Zick Y. Ser/Thr phosphorylation of IRS proteins: a molecular basis for insulin resistance. Sci STKE 2005 Jan. 25; 2005(268):e4.

Zimmet P, Alberti K G, Shaw J. Global and societal implications of the diabetes epidemic. Nature. 2001 Dec. 13; 414(6865):782-7.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for inhibiting follistatin comprising delivering to a patient an effective amount of a polynucleotide that suppresses the production of follistatin.

2. A method for inhibiting follistatin comprising delivering to a patient in need thereof a therapeutically-effective amount of a polynucleotide that suppresses the production of follistatin.

3. The method of claim 1 or 2 wherein the polynucleotide comprises an siRNA molecule.

4. The method of claim 1 or 2 wherein the polynucleotide further comprises a targeting agent.

5. The method of claim 4 wherein the targeting agent comprises an N-acetylgalactosamine (GalNAc) moiety.

6. The method of claim 2 wherein the patient has or is at risk for having a disease or condition selected from diabetes, pre-diabetes, metabolic syndrome, insulin resistance, dementia, and obesity.

7. The method of claim 6 wherein disease is prevented, treated, or ameliorated.

8. The method of claim 1 or 2 wherein the polynucleotide is delivered systemically.

9. The method of claim 7 wherein the polynucleotide is delivered intravenously.

10. The method of claim 1 wherein the polynucleotide comprises a DNA molecule encoding an siRNA molecule.

11. A method for delivering to a patient an effective amount of a compound that inhibits follistatin.

12. The method of claim 11, wherein the compound is selected from a group consisting of at least one of the following: an antibody, antibody fragment, FAb fragment, FAb′ fragment, nanobody, small molecule, polynucleotide, RNAi, siRNA.

13. A method for inhibiting follistatin comprising delivering to a patient an effective amount of a compound that suppresses the production of follistatin.

14. A method for inhibiting follistatin comprising delivering to a patient an effective amount of a compound that reduces the levels of follistatin.

15. A method for inhibiting follistatin comprising delivering to a patient an effective amount of a compound that inhibits the function of follistatin.

16. A method for treating a patient comprising delivering to a patient an effective amount of a compound that suppresses the production of follistatin.

17. A method for treating a patient comprising delivering to a patient an effective amount of a compound that inhibits follistatin.

18. A method of determining whether a compound is an inhibitor of Fst, comprising:
providing a Test Cell which overproduces Fst and exhibits an increase in binding of Fst to a protein, relative to a Control cell which produces Fst at a lower level, and which exhibits a lesser amount of binding of Fst to the protein;
exposing the Test Cell to the compound; and
measuring the amount of Fst bound to the protein.

19. The method of claim 18 wherein the compound which binds to Fst comprises an anti-Fst antibody or activin.

20. The method of claim 18 or 19 wherein the control cell does not produce a detectable level of Fst.

21. A method of identifying a compound capable of reducing the level of expression from an Fst promoter in a mammalian cell, comprising:
providing a Test Cell which contains the Fst promoter operably linked to a reporter gene such that increased expression of the Fst promoter sequence using a substance known to upregulate an endogenous Fst gene results in an increase in reporter protein levels;
exposing the Test Cell to the compound; and
determining whether an increase in reporter protein level in the Test Cell has occurred.

22. A method for identifying a compound that interferes with the ability of a Fst protein to promote insulin resistance, comprising:
providing a Test Cell which expresses IRS1 and the p110 catalytic subunit of PIK3 ;
exposing the Test Cell to serum comprising the Fst protein;
exposing the Test Cell to the compound; and
determining whether an increase in the interaction of the IRS1 with the p110 catalytic subunit has occurred.

23. A method for determining whether a compound is an inhibitor of Fst, comprising:
providing a Test Cell which expresses AKT;
exposing the Test Cell to serum comprising the Fst protein;
exposing the Test Cell to the compound; and
determining whether an increase in the phosphorylation of the AKT has occurred.

24. A method for determining whether a compound is an inhibitor of Fst, comprising:
providing a Test Cell which expresses hormone-sensitive lipase (HSL);
exposing the Test Cell to serum comprising the Fst protein;
exposing the Test Cell to the compound; and
determining whether a decrease in the phosphorylation of the HSL has occurred.

25. The method of any one of claims 22-24 wherein the Test Cell is exposed to the serum after exposure to the compound.

26. The method of any one of claims 22-25 wherein the serum is from an insulin resistant LDKO-mouse.

27. The method of any one of claims 22-26 wherein the Test Cell is a differentiated 3T3L1-adipocyte.

28. The method of any one of claims 18-27 wherein the compound is a small molecule

29. The method of any one of claims 18-27 wherein the compound comprises a protein.

30. The method of any one of claims 18-29 wherein the protein comprises an antibody.

31. A method of identifying a polynucleotide that reduces expression of Fst, comprising:
providing a Test Cell which produces Fst;
exposing the Test Cell to the polynucleotide; and
measuring the amount of the Fst in the cell.

32. The method of claim 31 wherein the polynucleotide comprises a double-stranded RNA molecule.

33. The method of claim 31 wherein the polynucleotide comprises a single-stranded RNA molecule.

34. The method of any one of claims 31-33 wherein the RNA molecule comprises at least 19 consecutive nucleotides that are complementary to a coding region encoding the Fst protein.

35. The method of any one of claims 18-34 wherein the Test Cell is a mammalian cell.

36. A method of restoring or enhancing insulin sensitivity in a cell comprising reducing or inhibiting Fst function.

37. The method of claim 36 wherein the cell is in vitro.

38. A method of treating a disease characterized by increased expression or activity of Fst, comprising reducing in a subject expression or activity of the Fst.

39. The method of claim 38 wherein the disease is a metabolic disease, diabetes, obesity, or a combination thereof.

40. The method of any one of claims 18-39 wherein the Fst is Fst288, Fst303, or Fst315.


SEQUENCE LISTING (1 sequence)

SEQ ID NO: 1 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW